(19) United States
(12) Patent Application Publication
(10) Pub. No.: US 2006/0079540 A1
(43) Pub. Date: Apr. 13, 2006

(54) Pde4 and pde3/4 inhibitors for use in the treatment of cachexia

(75) Inventor: Mathias Schmidt, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/535,815

(22) PCT Filed: Nov. 26, 2003
(86) PCT No.: PCT/EP03/13313

§ 371 Date: May 20, 2005

(30) Foreign Application Priority Data
Nov. 27, 2002 (EP) 02026548.4

(51) Int. Cl. A61K 31/522; A61K 31/513; A61K 31/473; A61K 31/426; A61K 31/277

(52) U.S. Cl. 514/263.34; 514/269; 514/263.35; 514/369; 514/521; 514/469; 514/416; 514/339; 514/290

(57) ABSTRACT

The invention relates to the use of a PDE4 or PDE3/4 inhibitor for the treatment of cachexia.

### FIELD OF APPLICATION OF THE INVENTION

The present invention relates to PDE4 inhibitors and PDE3/4 inhibitors for use in the treatment of cachexia. The substances used in accordance with this invention are known active compounds from the PDE4 inhibitor and PDE3/4 inhibitor class.

### PRIOR ART

WO9923076, WO0009504, WO0147914, WO0157036, WO02060898, U.S. 20020156105, U.S. Pat. No. 6,313,156, U.S. Pat. No. 5,728,844, EP1229034 list therapeutically active compounds useful as PDE4 Inhibitors as well as their use for the treatment of numerous diseases.

### DESCRIPTION OF THE INVENTION

Cachexia is a syndrome of wasting associated with many forms of cancer and chronic diseases, e.g. liver cirrhosis, chronic kidney insufficiency, COPD (chronic obstructive pulmonary disease), or chronic cardiac insufficiency. The clinical picture of cachexia includes weight loss, anorexia (i.e. loss of appetite and ability to eat), loss of protein mass and loss of fat mass, muscle atrophy, gain in the proportion of body-water, and a variety of metabolic changes. Generally, cachexia associated with cancer is not a local effect of a tumor, but is thought to arise from distant metabolic effects, i.e. it is a type of paraneoplastic syndrome. Clinical trials directed towards increasing energy intake have failed to reverse the symptoms of cachexia. The pattern of weight loss in cancer cachexia patients is different from normal starvation. The normal adaptive response to nutrient deprivation is to draw on energy-dense lipid while sparing protein, resulting in loss of fat and relative preservation of lean body tissue. In contrast, cachectic patients experience severe and incapacitating muscle wasting with relative sparing of adipose tissue.

Undisputedly, there is a medical need for better treatment options of cachexia, in particular in patients suffering from cachexia as a result of cancer. Cachexia is seen in more than 60% of cancer patients, and the effectiveness of cancer therapy is very often dependent on the presence or absence of cachexia symptoms. Shorter survival periods and poorer response to chemo- and radiation therapy are observed in patients with symptoms of cachexia [De Wys et al., Am. J. Med 69: 491-497 (1980); Kern et al., J. Parenter. Enter. Nutr. 12: 286-298 (1988)]. Cachexia is one of the most important contributors that lead to loss of quality of life in cancer patients and dependence on managed care, and mortality.

In general, two different approaches have been undertaken to manage and to treat the symptoms of cachexia: Firstly, nutritional supplementation with improved diet or, secondly, drugs to increase appetite have been applied to patients. But—like megestrol acetate—drugs to increase appetite have generally been disappointing with megestrol acetate only being capable of restoring adipose body weight.

Although little is known about the precise mechanisms of cachexia, recent studies indicate that inappropriate production and release of cytokines such as TNF-α, Interleukin-1, Interleukin-6, and Interferon-γ are involved in the induction of cachexia. Knapp et al. (1991) observed elevated TNF levels coinciding with weight loss in advanced stage IV breast cancer patients [Knapp et al., Ann Clin. Biochem., 28: 480-486 (1991)]. Sherry et al. reported that antibodies to TNF could significantly reduce the loss of carcass protein and fat loss in a murine sarcoma model [Sherry et al., FASEB J. 3: 1956-1962 (1989)]. Fong et al. found that IL-1 was able to match TNF-α and LPS as inducers of anorexia and cachexia in rats [Fong et al., Am. J. Physiol. 256: R659 (1989)]. Matthys and Billiau reviewed that further cytokines, e.g. Leukemia Inhibitory Factor (LIF), Ciliary Neurotrophic Factor (CNTF) and Interferon-γ, are associated with cachexia [Mattys and Billiau, Nutrition 13, 763-770 (1997)].

These data indicate that multiple cytokines secreted from tumor and host tissues either alone or in combination are able to cause the metabolic changes associated with cachexia and finally to induce wasting.

It is the object of the present invention to make available a treatment of cachexia which fulfil the following conditions: (1) Suppression or neutralization of cytokines involved in induction of cachectic symptoms, and (2) influencing the bioactivity of several and not only of a single cytokine.

Surprisingly, it has now been found that the use of a PDE4 or a PDE3/4 inhibitor fulfils the abovementioned conditions.

In a first embodiment of this invention, there is provided the use of a PDE4 inhibitor or a PDE3/4 inhibitor or a pharmaceutically acceptable derivative thereof for the manufacture of a pharmaceutical composition for the treatment of cachexia.

According to this invention, “PDE4 inhibitor” refers to a selective phosphodiesterase (PDE) inhibitor, which inhibits preferentially the type 4 phosphodiesterase (PDE4) when compared to other known types of phosphodiesterase, e.g. type 1, 2, 3, 5, etc. (PDE1, PDE2, PDE3, PDE5, etc.). According to this invention, a selective PDE inhibitor preferentially inhibiting PDE4 refers to a compound having a lower IC₅₀ for PDE4 (i.e. the IC₅₀ for PDE4 inhibition is about 10 times lower than the IC₅₀ for inhibition of other known types of phosphodiesterase, e.g. type 1, 2, 3, 5, etc.) and therefore is more potent to inhibit PDE4. Analogously, the term “PDE3/4 inhibitor” is defined as a compound having a lower IC50 for the type 3/4 phosphodiesterases and therefore a PDE3/4 inhibitor is more potent to inhibit PDE3/4.

Methods to determine the activity and selectivity of a phosphodiesterase inhibitor are known to the person skilled in the art. In this connection it may be mentioned, for example, the methods described by Thompson et al. (Adv Cycl Nucl Res 10: 69-92, 1979), Giembycz et al. (Br J Pharmacol 118:1945-1958, 1996) and the phosphodiesterase scintillation proximity assay of Amersham Pharmacia Biotech.

Possible PDE4 or PDE3/4 inhibitors within the meaning of the present invention are those PDE4 or PDE3/4 inhibitors which are cited expressis verbis as an example, or described or claimed generically in the following patent applications and patents: DE 1545687, DE 2028869, DE 2123328, DE 2315801, DE 2402908, DE 2413935, DE 3900233, EP 0103497, EP 0139464, EP 0158380, EP 0163965, EP 0335386, EP 0389282, EP 0393500, EP 0428302, EP 0435811, EP 0449216, EP 0459505, EP 0470805, EP 0490823, EP 0506194, EP 0510562, EP 0511865, EP 0527117, EP 0553174, EP 0557016, EP 0626939, EP 0664289, EP 0671389, EP 0685474, EP 0685475, EP 0685479, EP 0731099, EP 0736532, EP 0738715, EP 0748805, EP 0763534, EP 0816357, EP 0819688, EP 0819689, EP 0832886, EP 0834508, EP 0848000, JP 92234389, JP 94329652, JP 95010875, JP 98072415, JP 98147585, U.S. Pat. No. 5,703,098, U.S. Pat. No. 5,739,144, WO 9117991, WO 9200968, WO 9212961, WO 9307146, WO 9315044, WO 9315045, WO 9318024, WO 9319068, WO 9319720, WO 9319747, WO 9319749, WO 9319751, WO 9325517, WO 9402465, WO 9412461, WO 9420455, WO 9422852, WO 9427947, WO 9500516, WO 9501338, WO 9501980, WO 9503794, WO 9504045, WO 9504046, WO 9505386, WO 9508534, WO 9509623, WO 9509624, WO 9509627, WO 9509836, WO 9514667, WO 9514680, WO 9514681, WO 9517392, WO 9517399, WO 9519362, WO 9520578, WO 9522520, WO 9524381, WO 9527692, WO 9535281, WO 9535283, WO 9535284, WO 9600218, WO 9601825, WO 9606843, WO 9603399, WO 9611690, WO 9611917, WO 9612720, WO 9631486, WO 9631487, WO 9635683, WO 9636595, WO 9636596, WO 9636611, WO 9636625, WO 9636626, WO 9636638, WO 9638150, WO 9639408, WO 9640636, WO 9703967, WO 9704779, WO 9705105, WO 9708143, WO 9709345, WO 9712895, WO 9718208, WO 9719078, WO 9720833, WO 9722585, WO 9722586, WO 9723457, WO 9723460, WO 9723461, WO 9724117, WO 9724355, WO 9725312, WO 9728131, WO 9730999, WO 9731000, WO 9732853, WO 9735854, WO 9736905, WO 9740032, WO 9743288, WO 9744036, WO 9744322, WO 9747604, WO 9748697, WO 9804534, WO 9805327, WO 9806692, WO 9806704, WO 9807715, WO 9808828, WO 9808830, WO 9808841, WO 9808844, WO 9809946, WO 9809961, WO 9811113, WO 9814448, WO 9818796, WO 9821207, WO 9821208, WO 9821209, WO 9822453, WO 9831674, WO 9840382, WO 9845268, WO 9855481, WO 9856756, WO 9905111, WO 9905112, WO 9505113, WO 9906404, WO 9918095, WO 9931071, WO 9931090, WO 9947505, WO 9957115, WO 9957118, WO 9964414, WO 0001695, WO 0012501, WO 0042017, WO 0042018, WO 0042019, WO 0042020, WO 0042034, WO 0119818, WO 0130766, WO 0130777, WO 0151470, WO 0206239, WO 0206270, WO 0205616 and WO 0206238.

In addition, PDE4 and PDE3/4 inhibitors are exemplary exhibited on the following pages with the aid of their formulae:

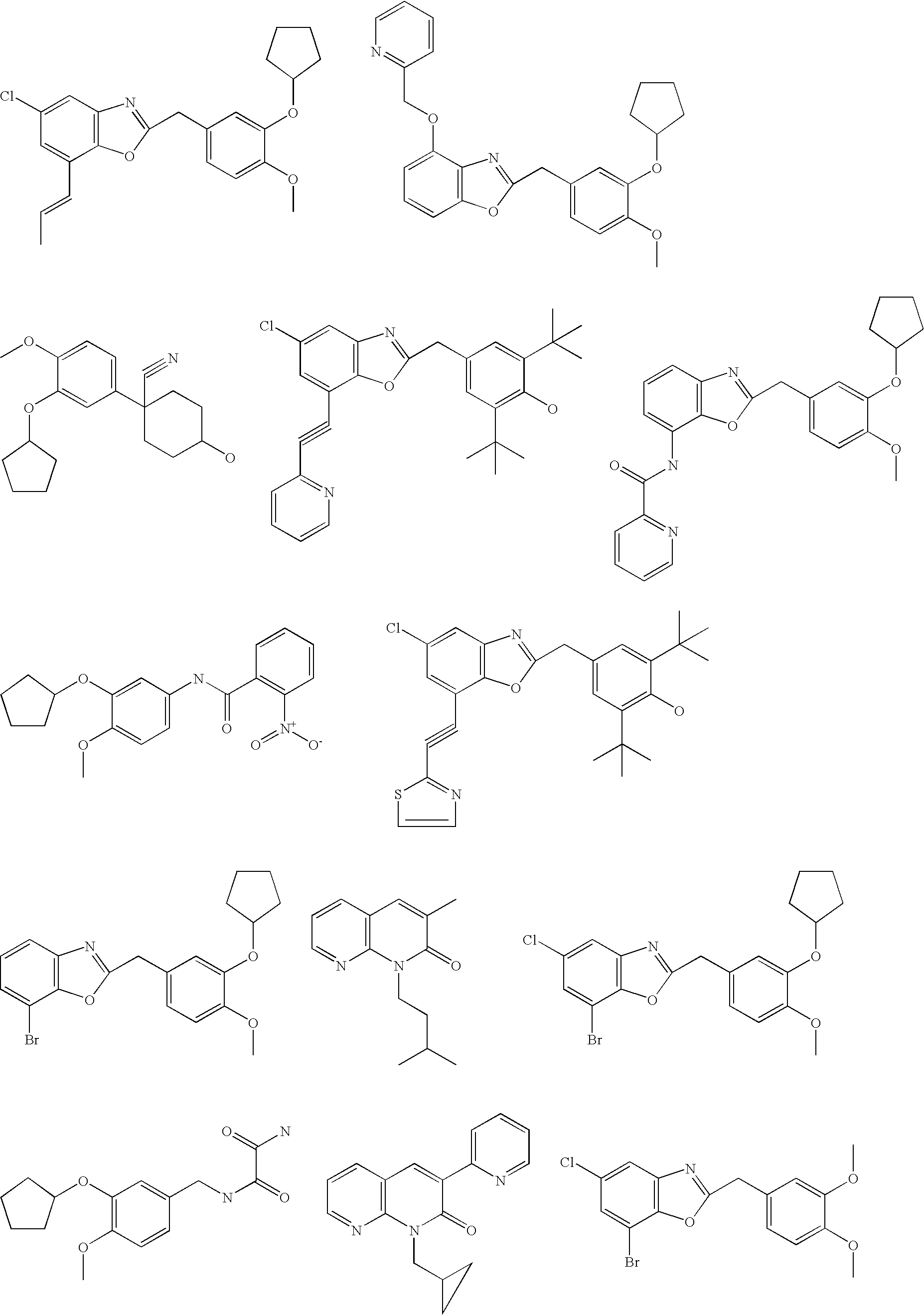

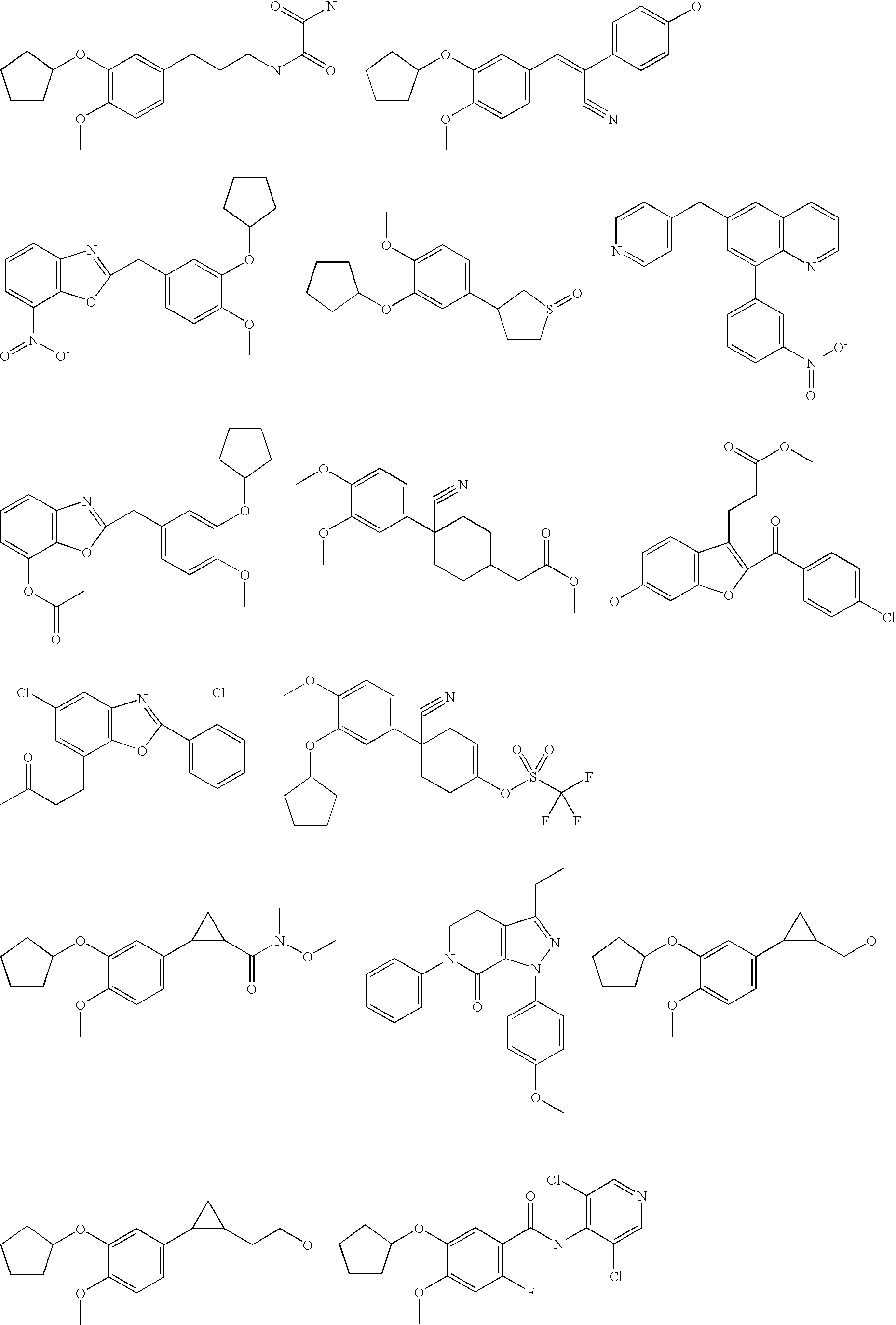

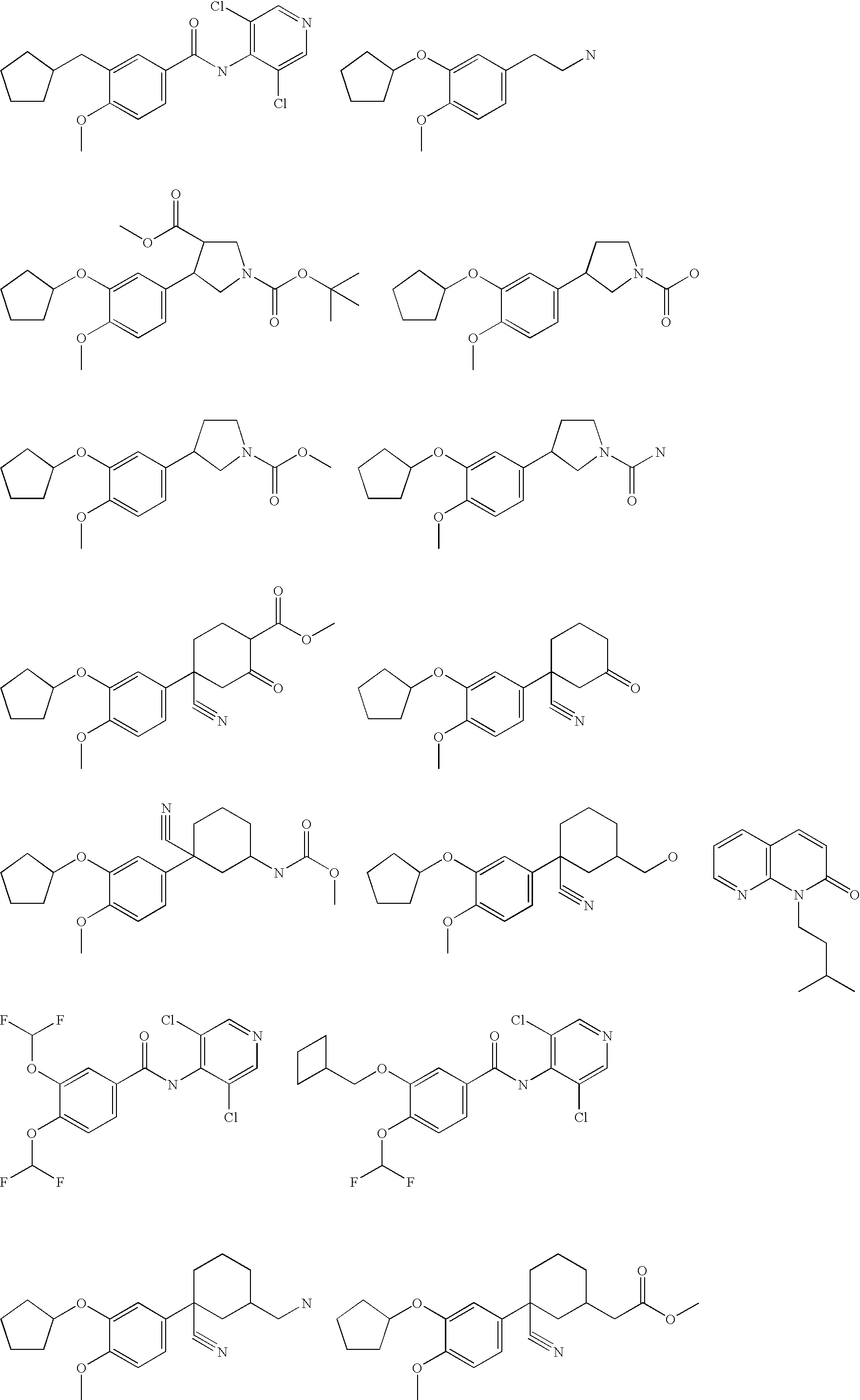

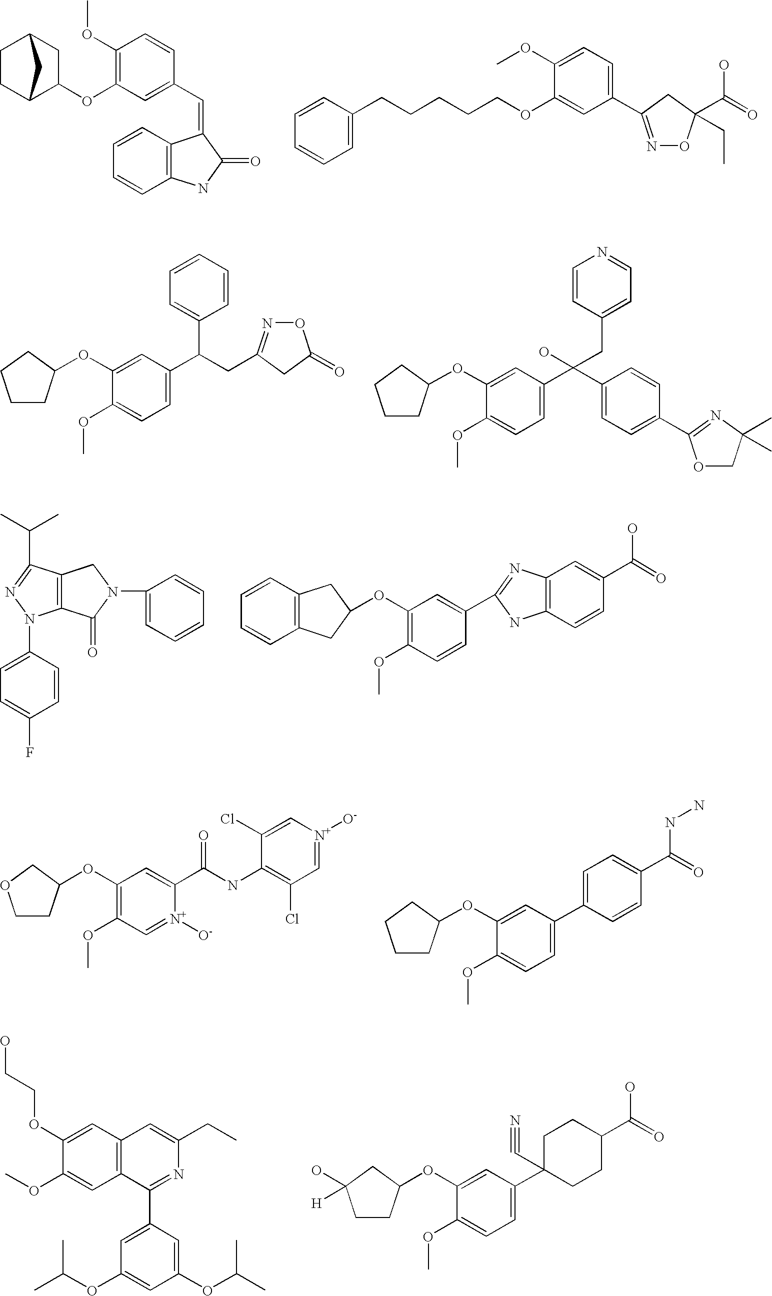

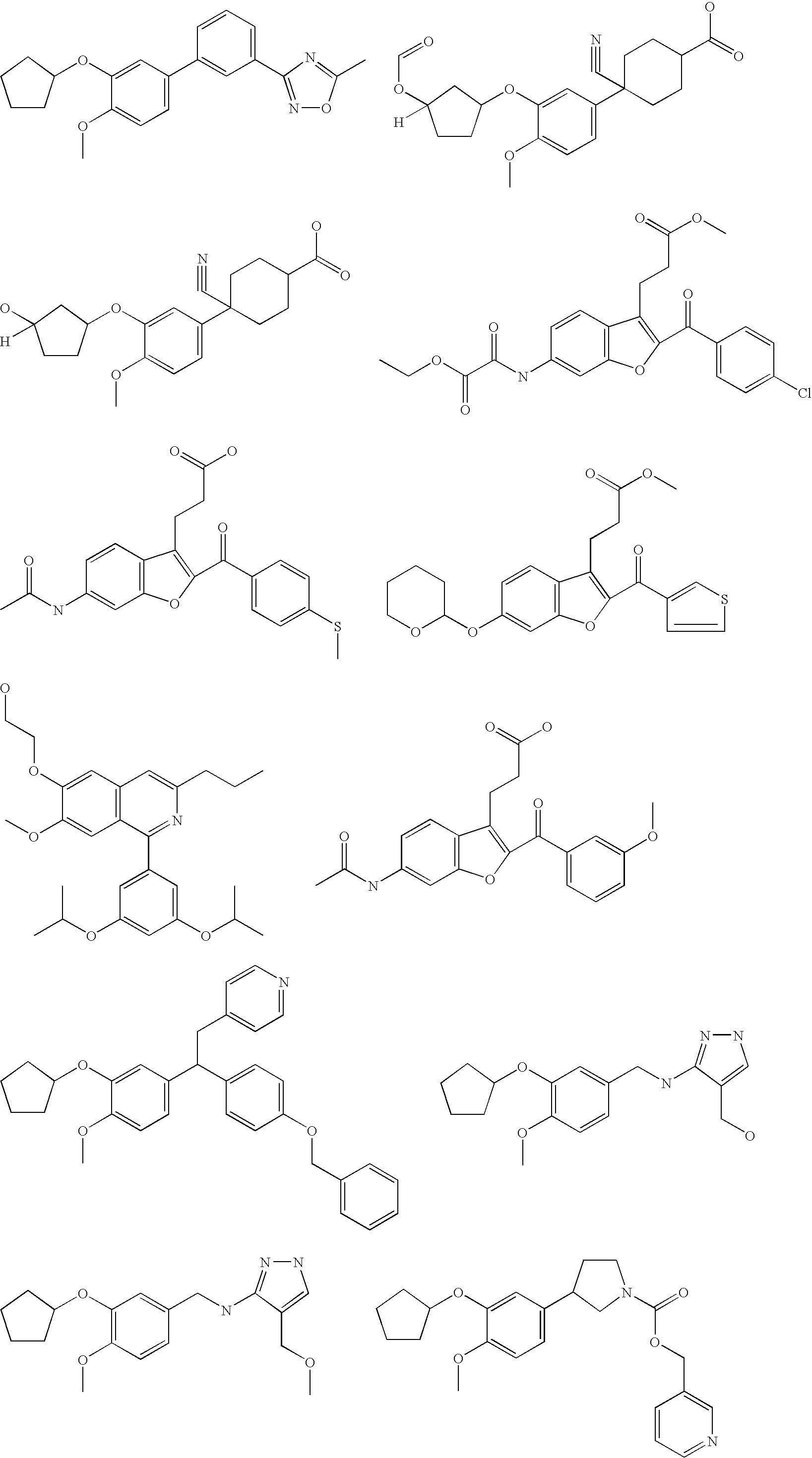

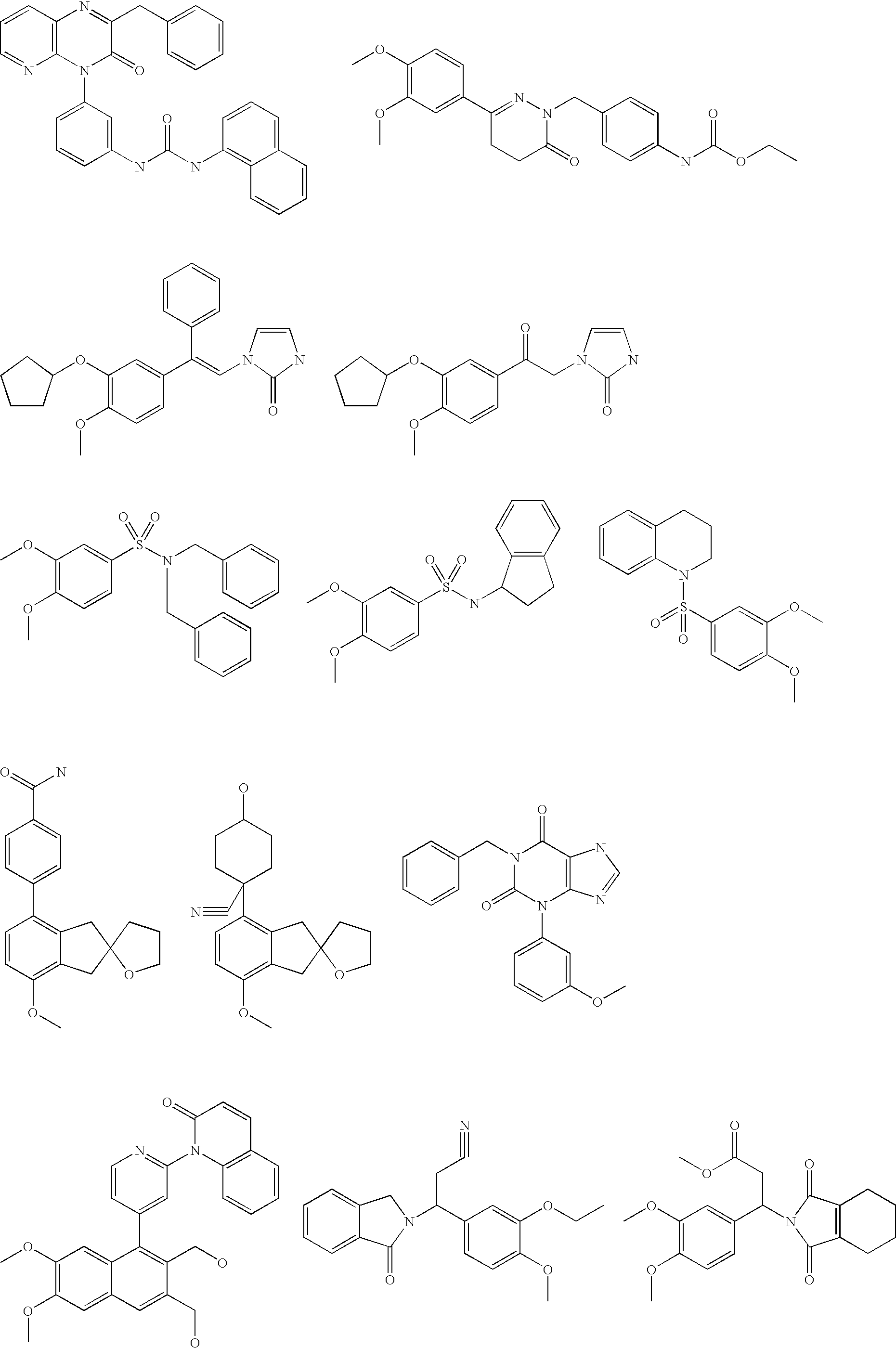

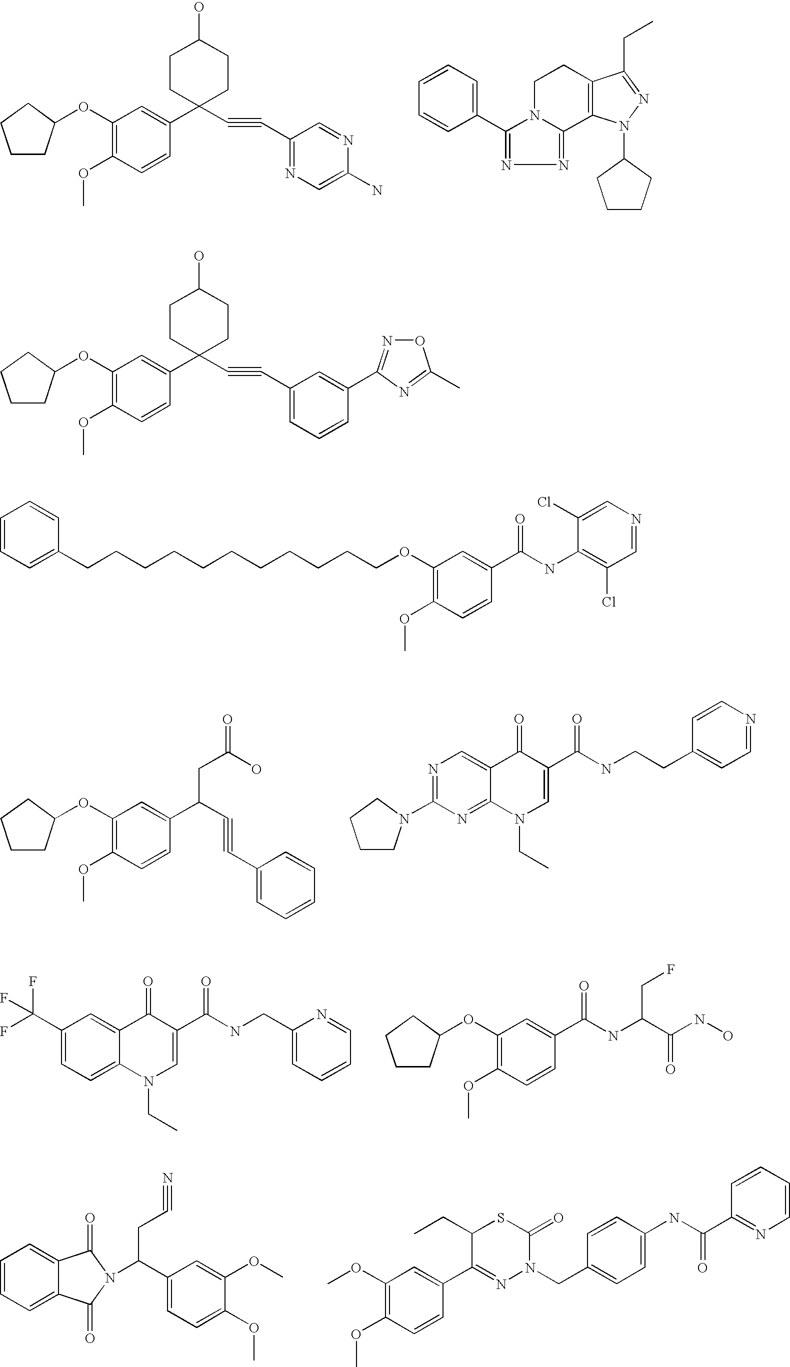

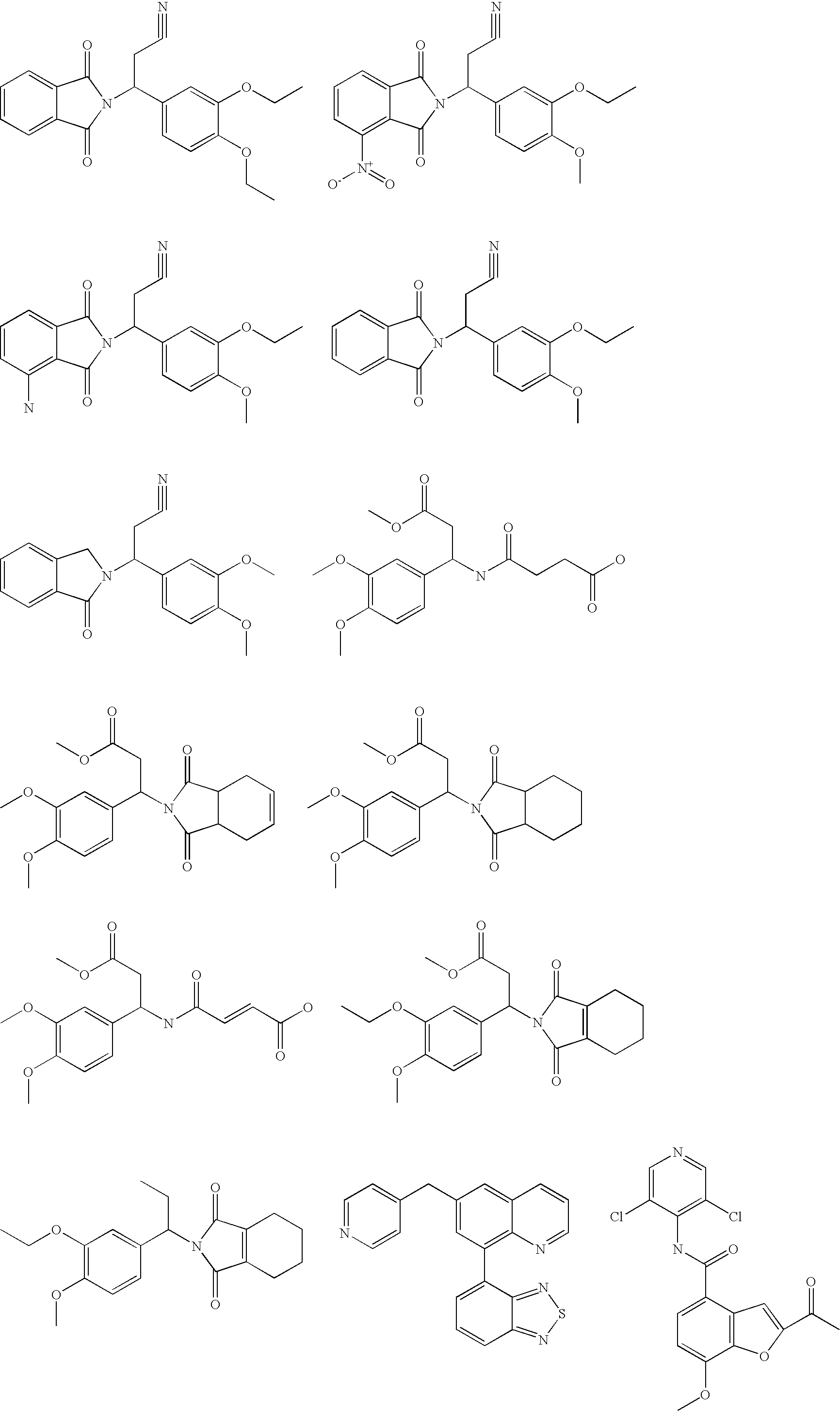

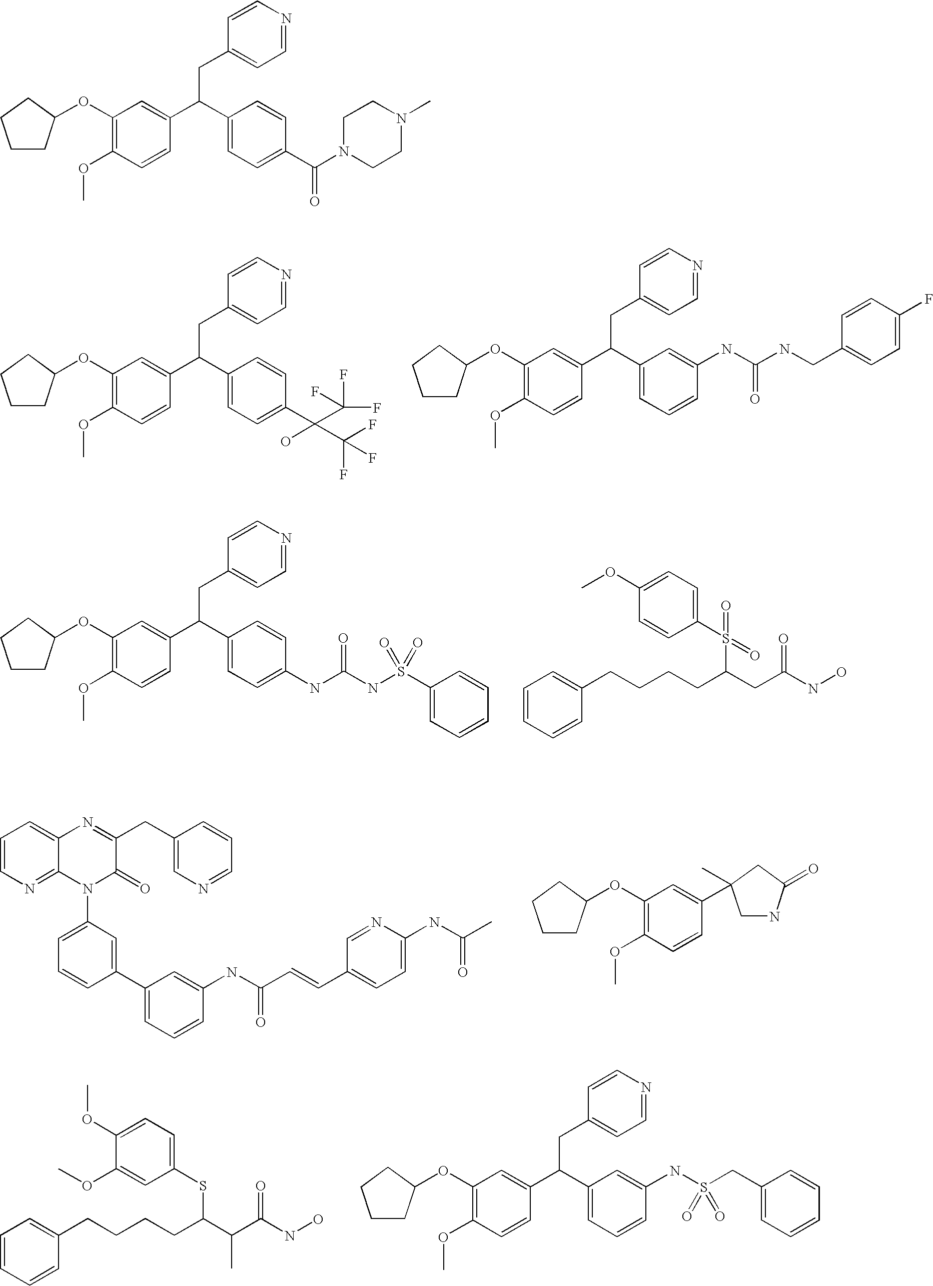

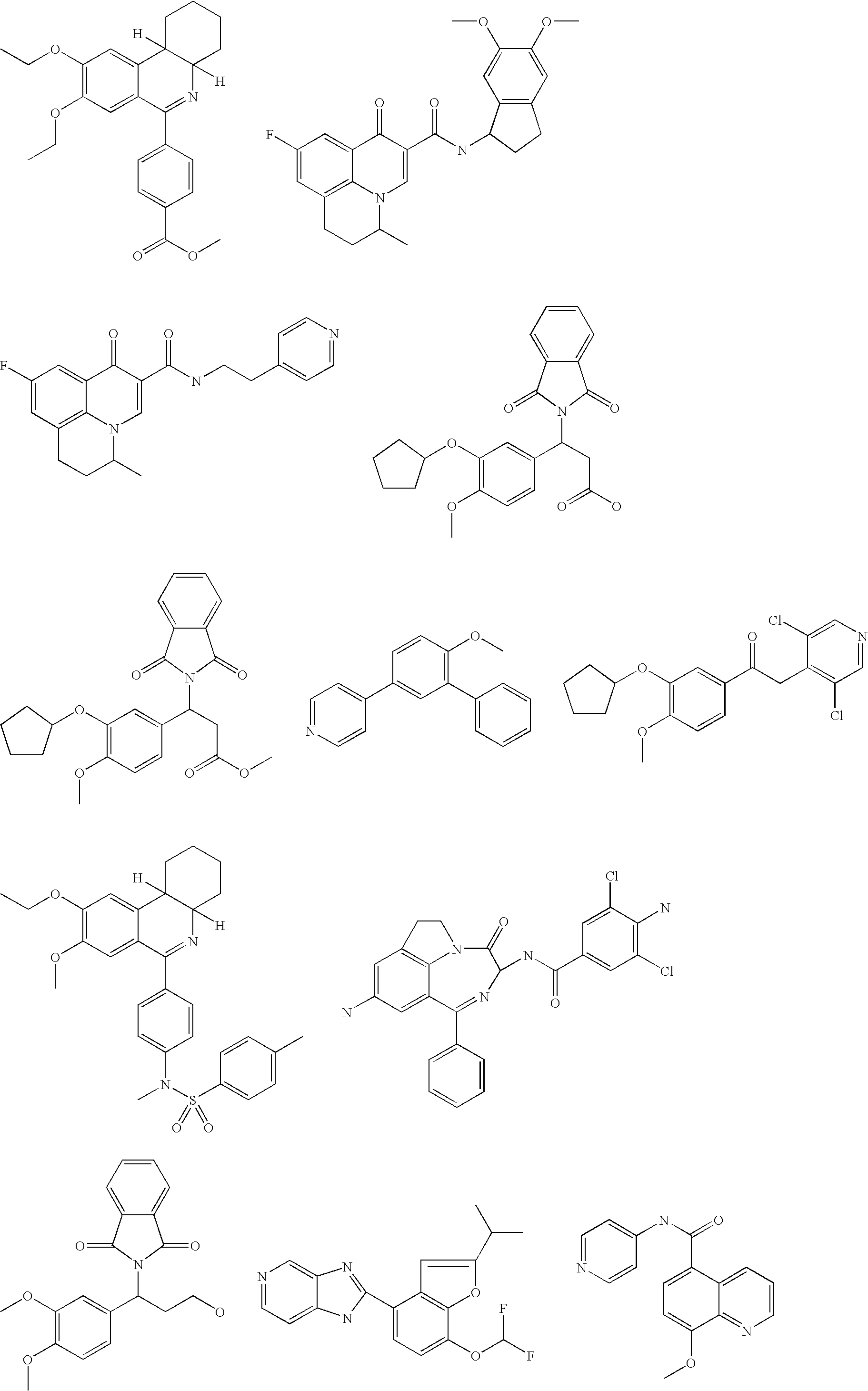

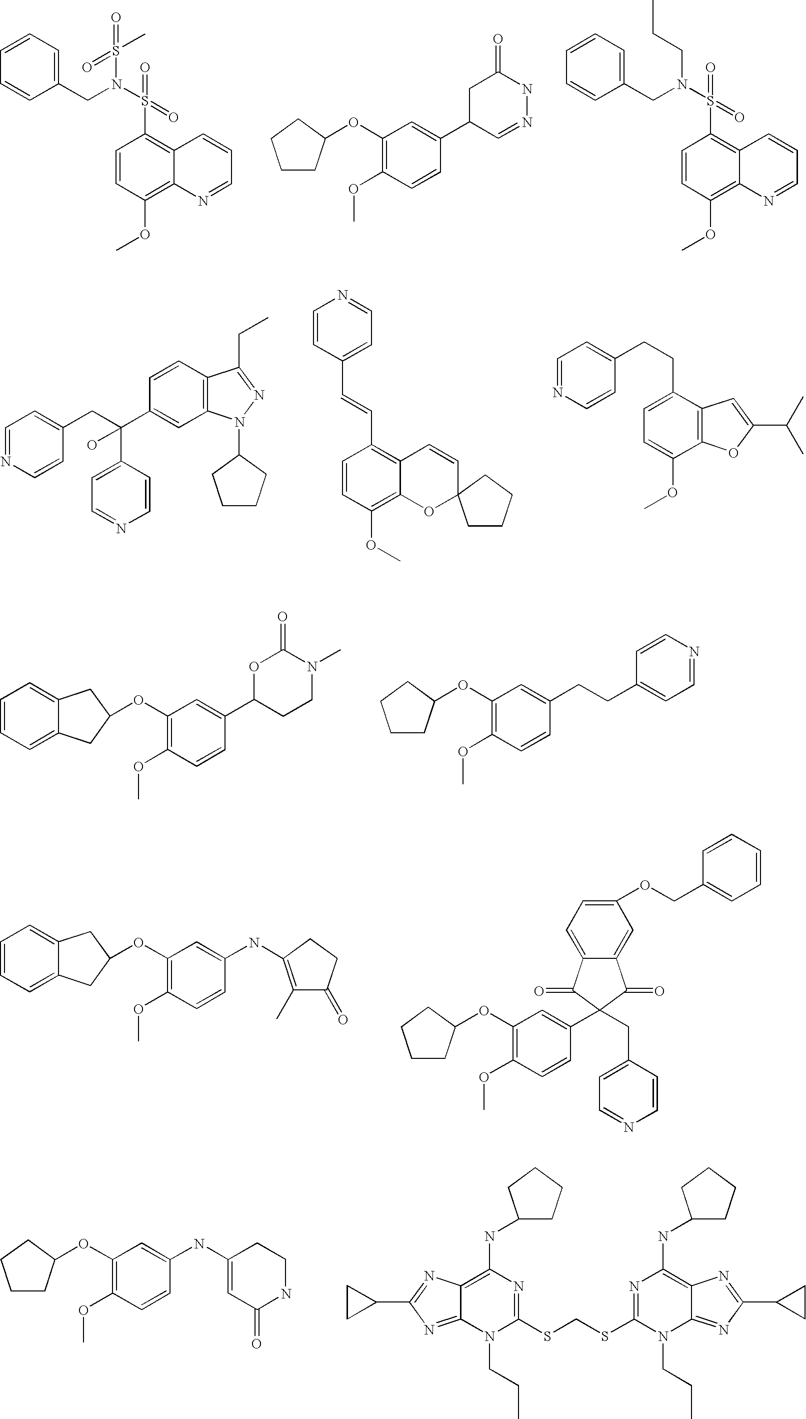

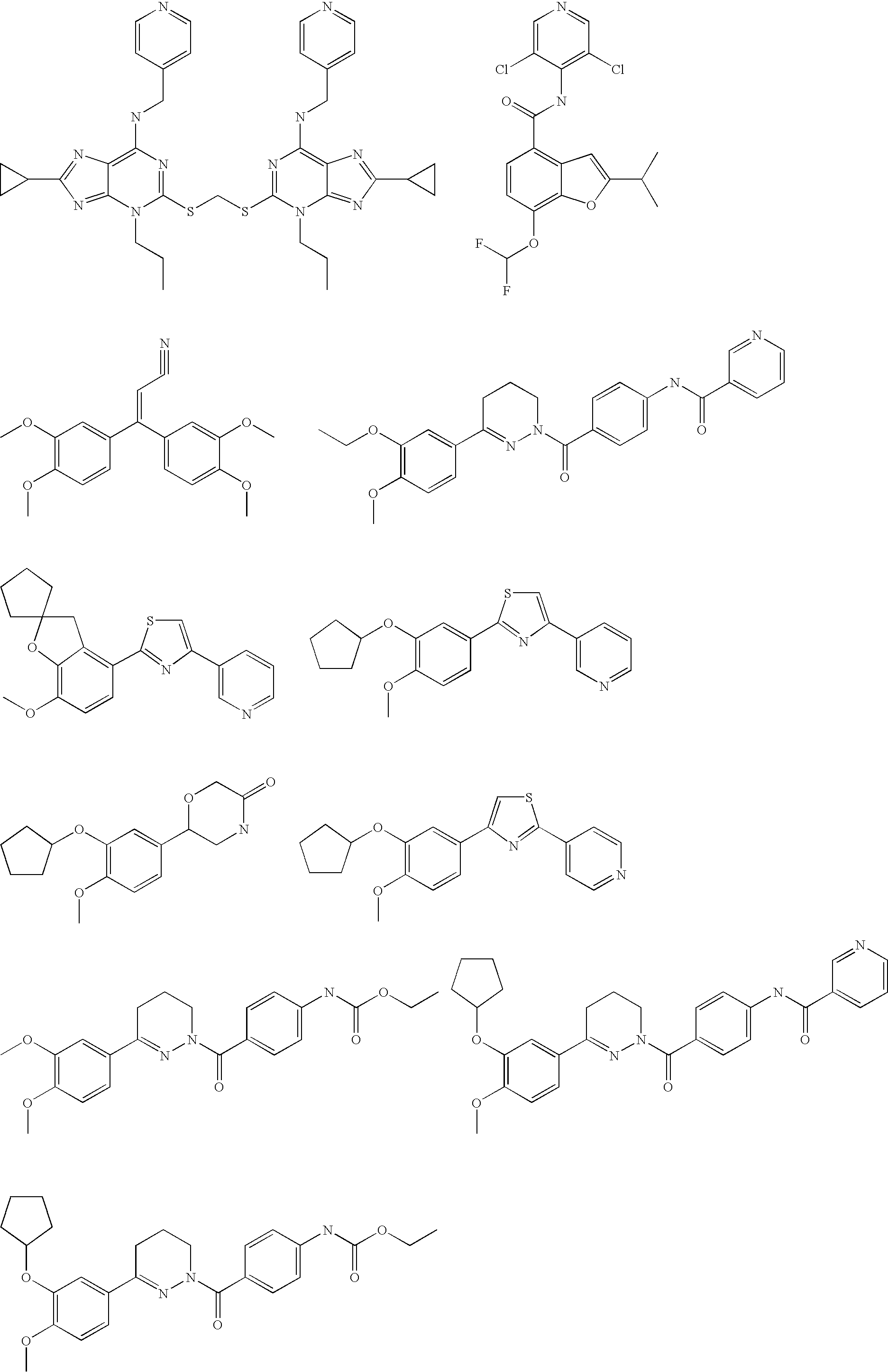

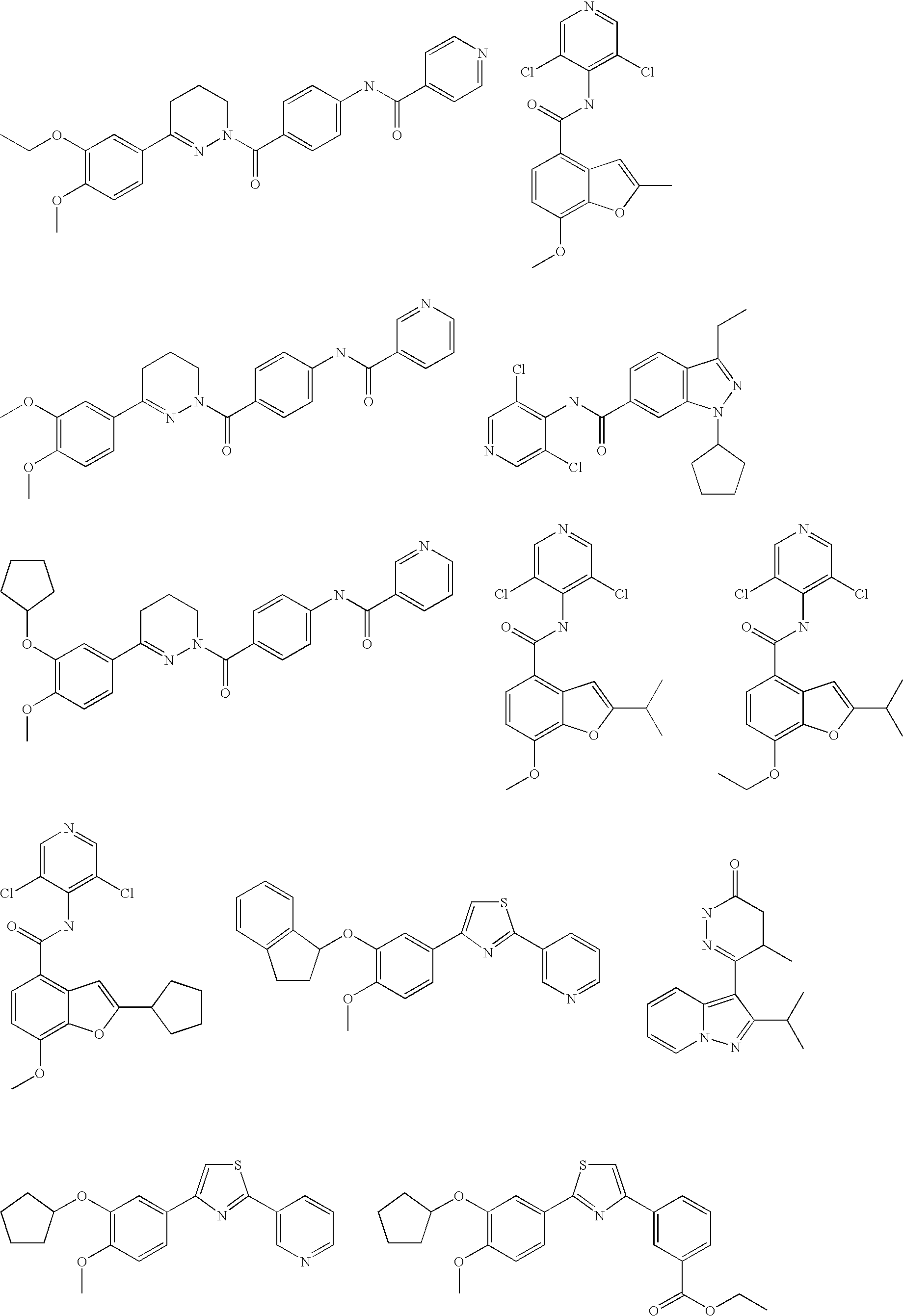

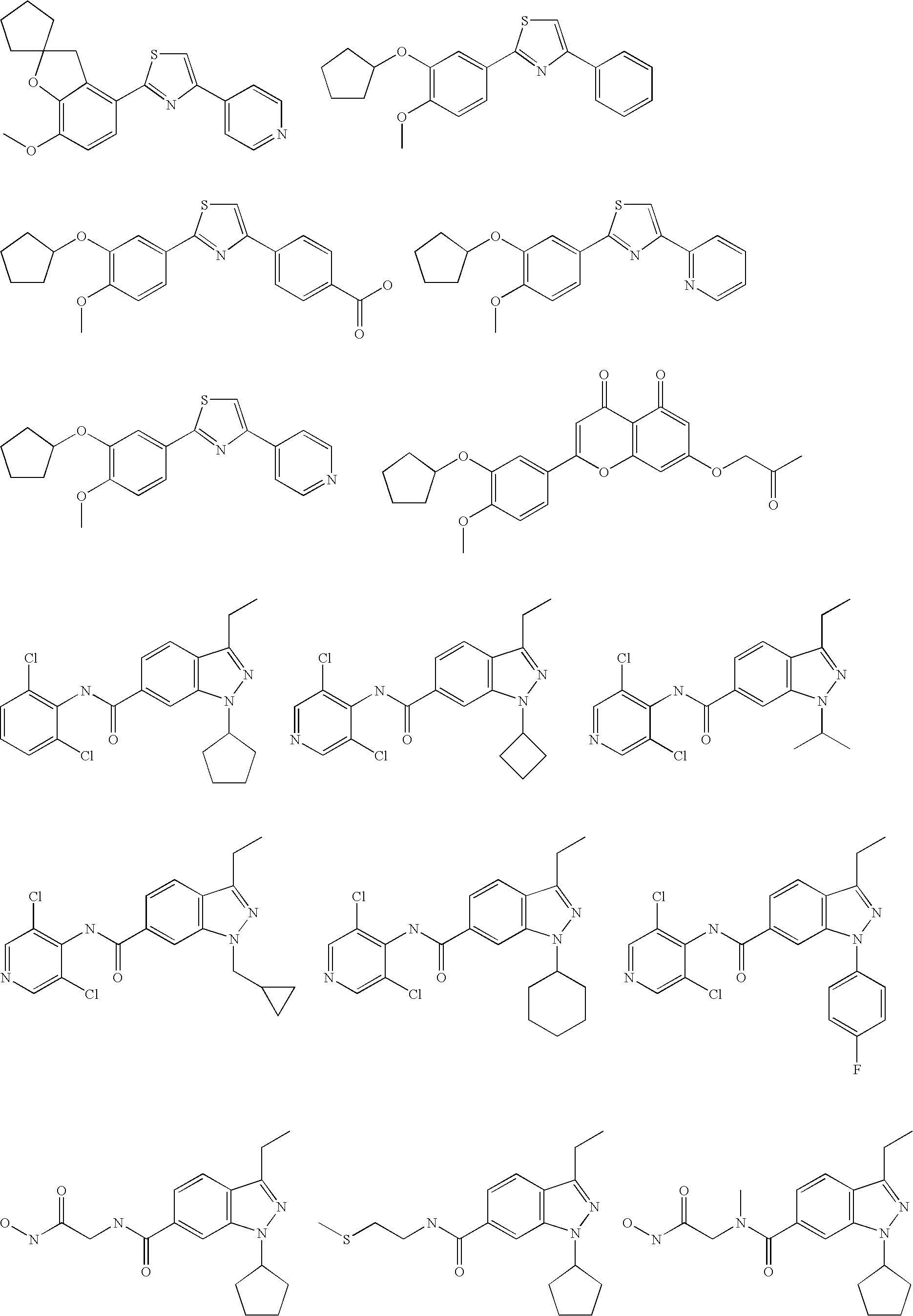

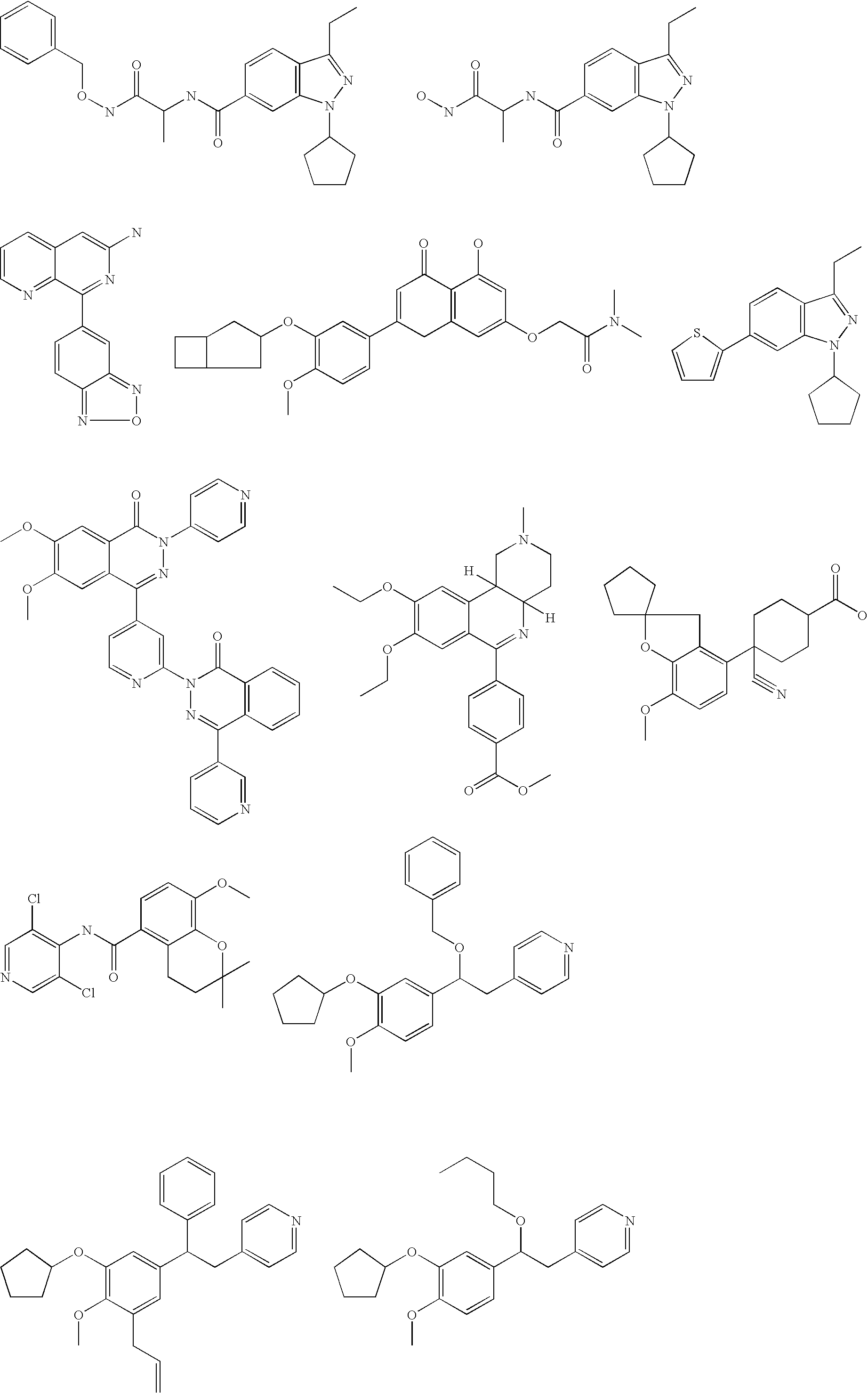

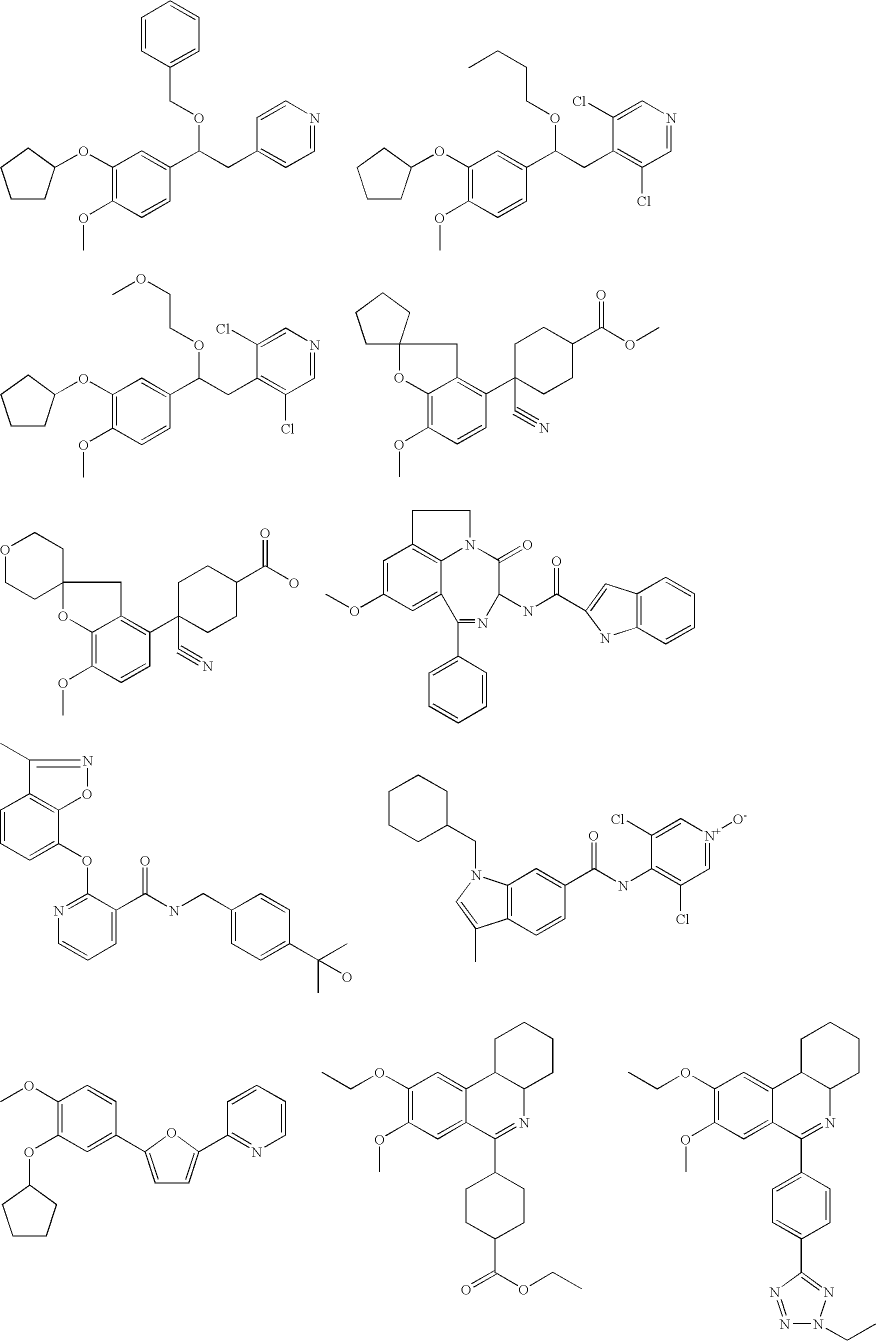

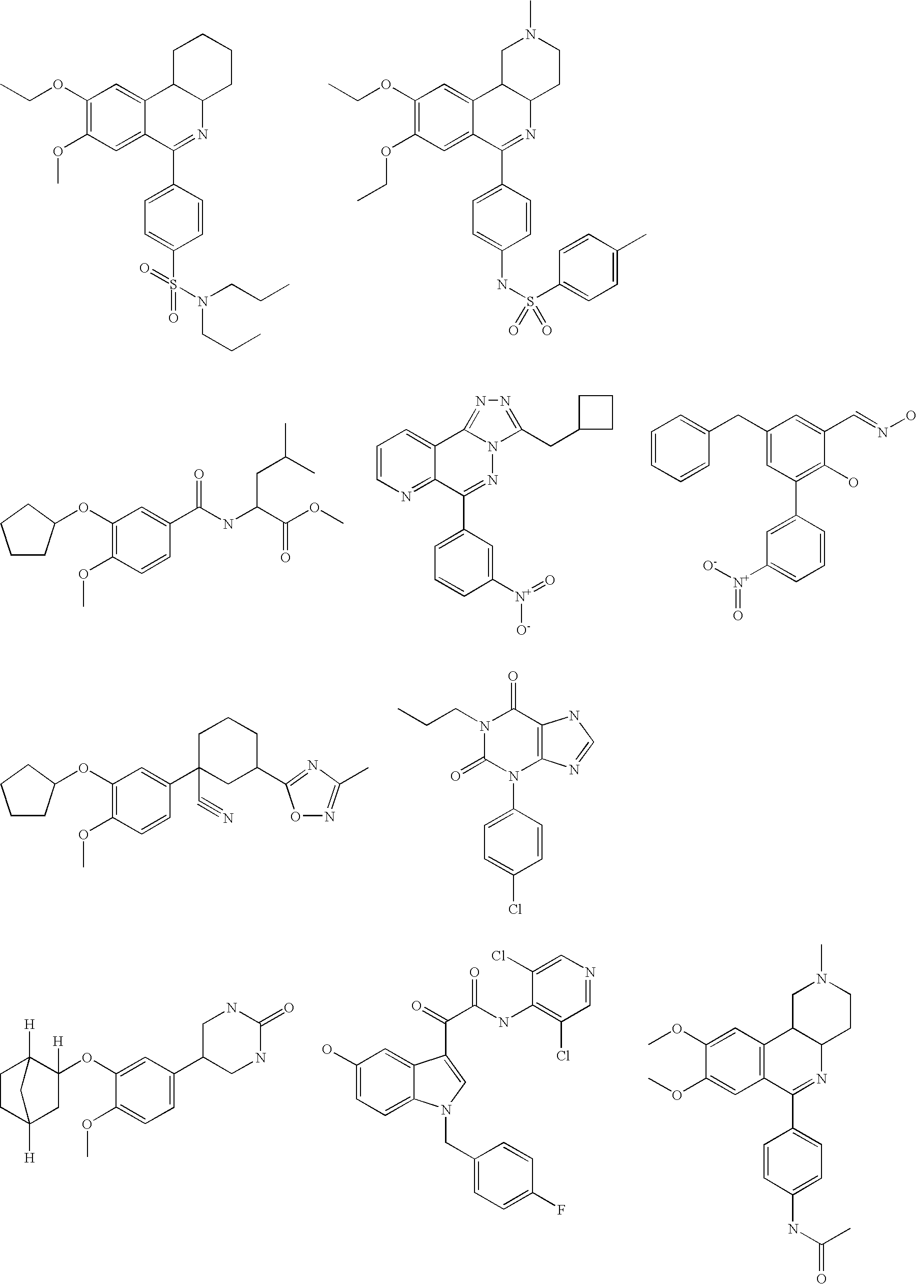

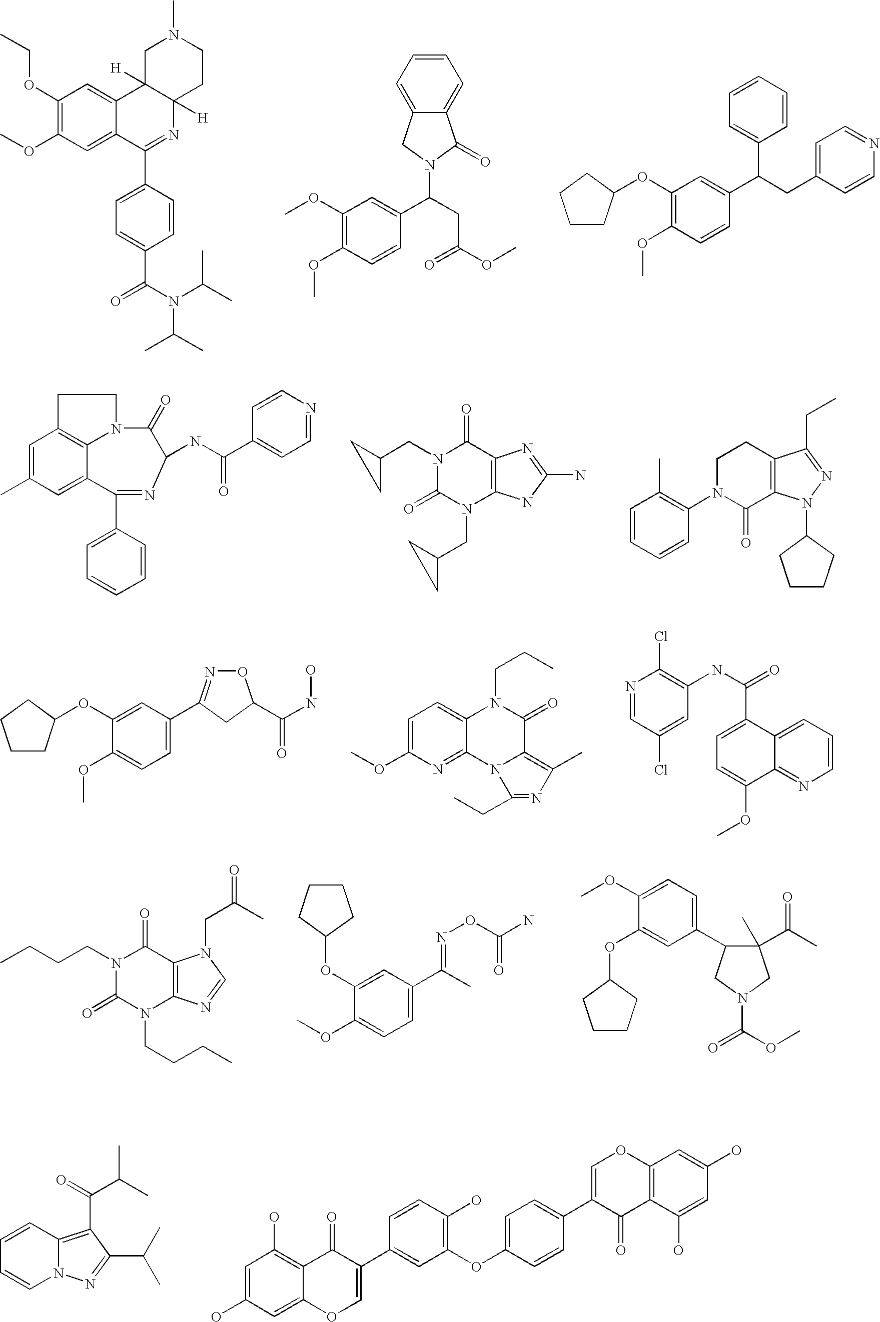

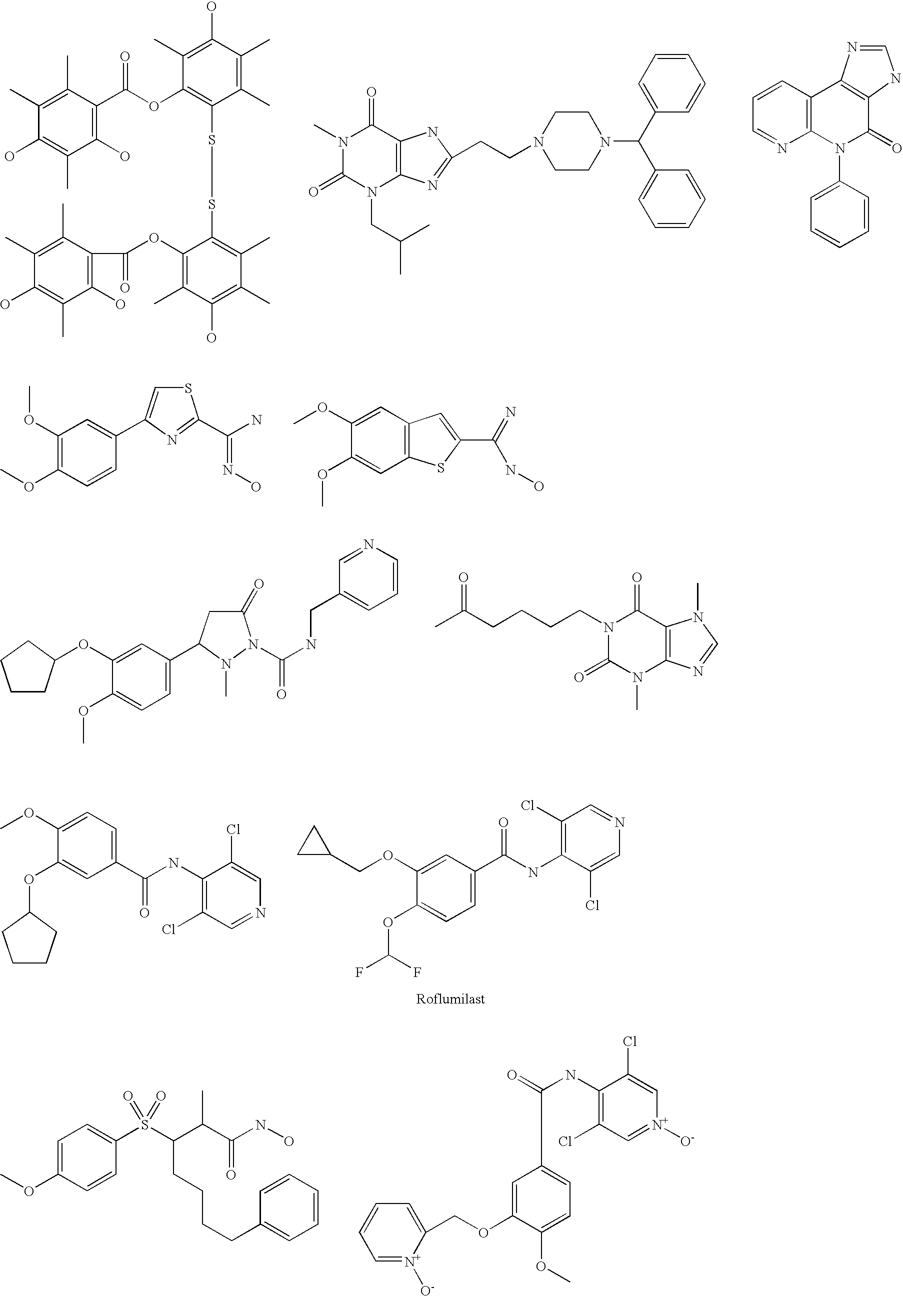

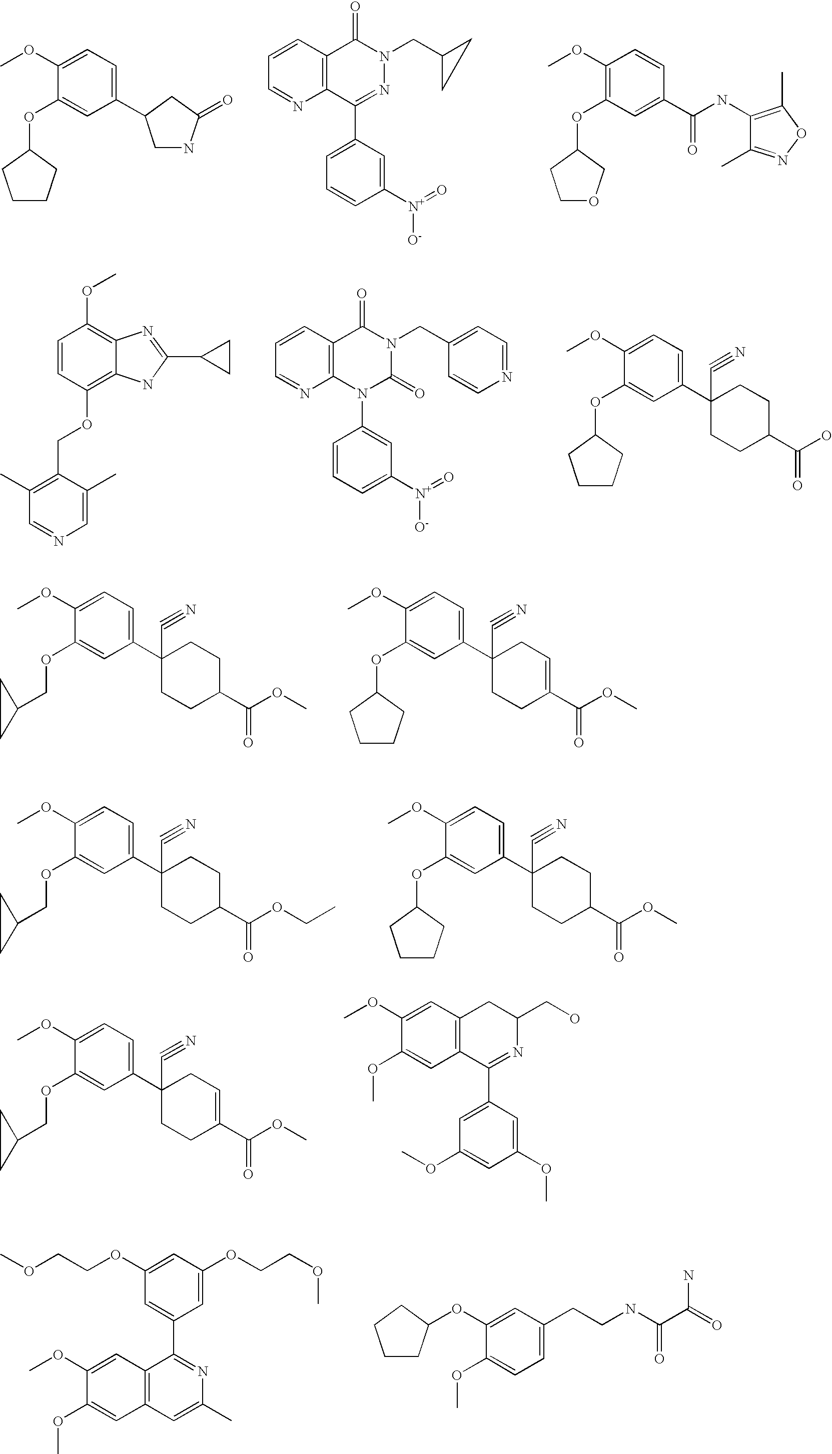

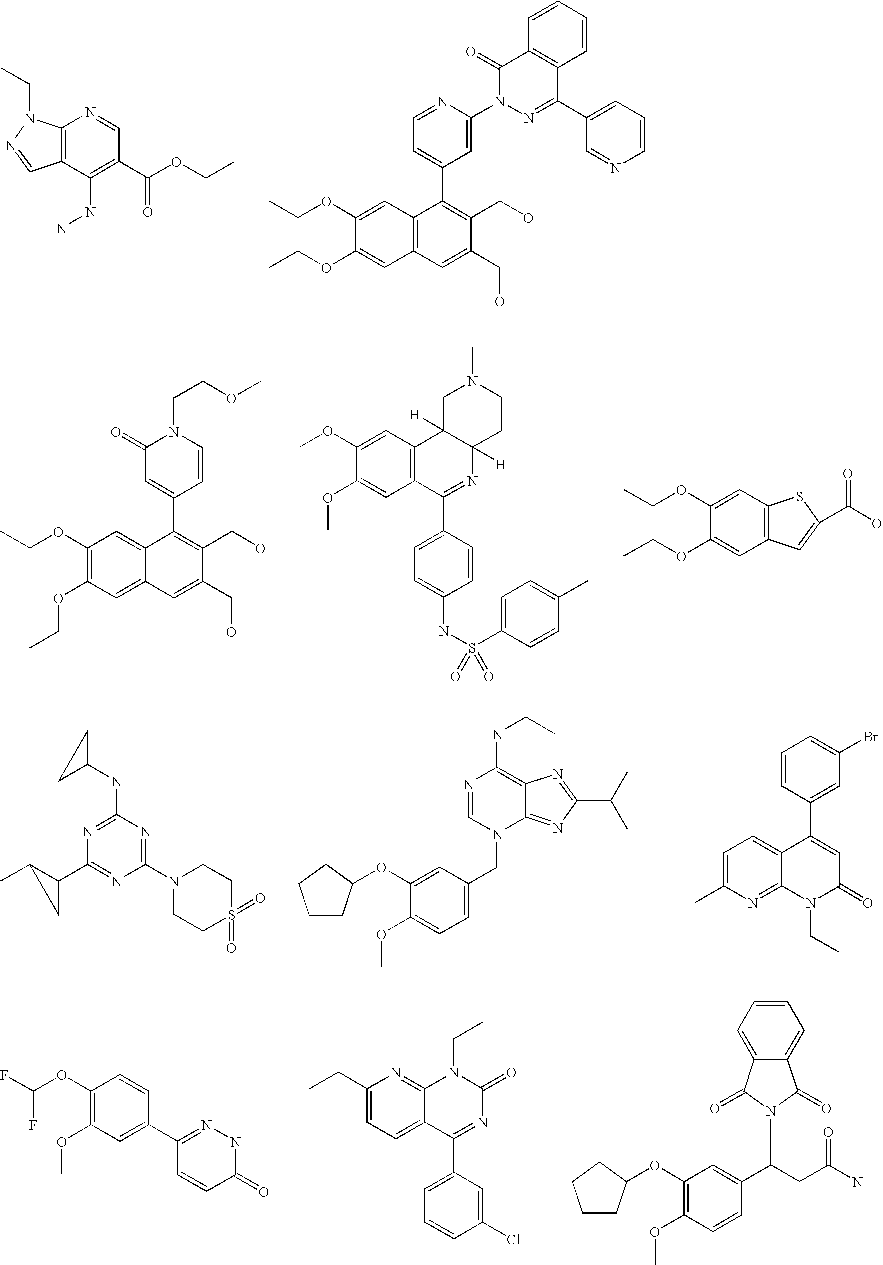

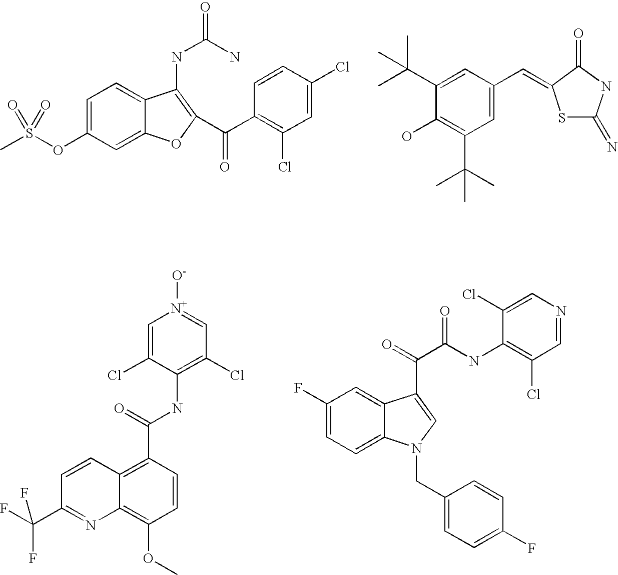

In the above cited formulae there is given neither any stereochemical information nor are hydrogen atoms indicated [—O is accordingly —OH, —N is NH, —N is NH₂. Methyl groups, e.g. on the oxygen atoms, are indicated by lines].

Furthermore, those PDE4 inhibitors and PDE3/4 inhibitors are preferred which are cited expressis verbis as an example and/or claimed generically in the patent applications or patents EP 0163965, EP 0389282, EP 0393500, EP 0435811, EP 0482302, EP 0499216, EP 0506194, EP 0510562, EP 0528922, EP 0553174, EP 0731099, WO 9319749, WO 9500516, WO 9501338, WO 9600218, WO 9603399, WO 9611690, WO 9636625, WO 9636626, WO 9723457, WO 9728131, WO 9735854, WO 9740032, WO 9743288, WO 9809946, WO 9807715, WO 9808841, WO 9821207, WO 9821208, WO 9821209, WO 9822453, WO 9831674, WO 9840382, WO 9855481, WO 9905111, WO 9905112, WO 9905113, WO 9931071, WO 9931090, WO 9947505, WO 9957115, WO 9957118, WO 9964414, WO 0001695, WO 0012501, WO 0042017, WO 0042018, WO 0042019, WO 0042020, WO 0042034, WO 0119818, WO 0130766, WO 0130777, WO0151470, WO 0206239, WO 0206270, WO 0205616 and WO 0206238 and the compounds with the following research codes: CDC-998, D-4396, SCH-351591, IC-485, CC-1088 and KW-4490. Substances having good oral availability are preferred here.

More particularly preferred PDE4 inhibitors or PDE3/4 inhibitors are the compounds with the research codes CDC-998, SH-636, D-4396, SCH-351591, IC-485, CC-1088, KW-4490 and 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1 jk][1,4]benzo-diazepin-3(R)-yl]pyridine-4-carboxamide [Research-Code: CI-1018], 4-(3,4-dimethoxyphenyl)thiazole-2-carboxamideoxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a, 10b-hexahydro-6-(4-diisopropylaminocarbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], γ-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [Research-Code: BAY-19-8004], (Z)-5-(3,5-di-tertbutyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST] and 3-Cyclopropylmethoxy-4-difluoromethoxy-N-(3,5-dichloropyrid-4-yl)-benzamide [INN: ROFLUMILAST].

Most particularly preferred PDE4 inhibitors or PDE3/4 inhibitors are 3-Cyclopropylmethoxy-4-difluoro methoxy-N-(3,5-dichloropyrid-4-yl)-benzamide [INN: ROFLUMILAST] and (−)-cis-9-ethoxy-8-methoxy-2-methyl-1,2,3,4,4a, 10b-hexahydro-6-(4-diisopropylaminocarbonylphenyl)-benzo-[c][1,6]naphthyridine [INN: PUMAFENTRINE]. The PDE 4 inhibitor N-(3,5-dichloropyrid-4-yl)-3-cyclopropylmethoxy-4-difluoromethoxybenzamide (INN: ROFLUMILAST) and its N-oxide is described in WO95/01338.

In a further embodiment of this invention, there is provided the use of (−)-cis-9-ethoxy-8-methoxy-2-methyl-1,2,3,4,4a, 10b-hexahydro-6-(4-diisopropylaminocarbonylphenyl)-benzo-[c][1,6]naphthyridine [INN: PUMAFENTRINE] or a pharmaceutically acceptable derivative thereof for the manufacture of a pharmaceutical composition for the treatment of cachexia.

In a further embodiment of this invention, there is provided the use of 3-Cyclopropylmethoxy-4-difluoromethoxy-N-(3,5-dichloropyrid-4-yl)-benzamide [INN: ROFLUMILAST] or a pharmaceutically acceptable derivative thereof for the manufacture of a pharmaceutical composition for the treatment of cachexia.

In the context of the present invention, unless otherwise stated, a pharmaceutically acceptable derivative of an active ingredient means a pharmaceutically acceptable salt or solvate (e.g. hydrate), a pharmaceutically acceptable solvate of such salt, a pharmaceutically acceptable N-oxide or a pharmaceutically acceptable salt or solvate of the latter.

According to this invention, suitable pharmaceutically acceptable salts refer to water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid, the acids being employed in salt preparation depending on whether it is a mono- or polybasic acid and depending on which salt is desired—in an equimolar quantitative ratio or one differing therefrom. Furthermore, the active compounds mentioned can also be present as pure enantiomers or as enantiomer mixtures in any mixing ratio.

In addition, suitable pharmaceutically acceptable salts also refer to salts with bases, e.g. alkali metal (lithium, sodium, potassium) or calcium, aluminium, magnesium, titanium, ammonium, meglumine or guanidinium salts, which also employ bases in salt preparations in an equimolar quantitative ratio or deviations of it.

PDE4 inhibitors and PDE3/4 inhibitors used in the present invention are capable of existing in stereoisomeric forms. The invention encompasses all stereoisomers of PDE4 inhibitors and PDE3/4 inhibitors and mixtures thereof including racemates. Tautomers of PDE4 inhibitors and PDE3/4 inhibitors and mixtures thereof are also part of the present invention.

In a further embodiment of this invention, there is provided the use of 3-Cyclopropylmethoxy-4-difluoromethoxy-N-(3,5-dichloropyrid-4-yl)-benzamide [INN: ROFLUMILAST] or a pharmaceutically acceptable salt or solvate (e.g. hydrate) thereof, or a pharmaceutically acceptable solvate of such salt, or a pharmaceutically acceptable N-oxide thereof or a pharmaceutically acceptable salt or solvate of the latter for the manufacture of a pharmaceutical composition for the treatment of cachexia.

According to this invention, treatment refers to the administration of a PDE4 inhibitor or a PDE3/4 inhibitor or a pharmaceutically acceptable derivative thereof in a human, whereby the activity of said PDE4 inhibitor or PDE3/4 inhibitor or pharmaceutically acceptable derivative thereof results in suppression or neutralization of cytokines involved in induction of cachectic symptoms or in influencing the bioactivity of several cytokines. According to this invention, treatment also refers to prophylaxis which itself refers to measures designed to prevent the occurrence of disease or its dissemination.

In a further embodiment of this invention, there is provided the use of a PDE4 inhibitor or PDE3/4 inhibitor or a pharmaceutically acceptable derivative thereof for the manufacture of a pharmaceutical composition for the treatment of cachexia as a result of cancer, chronic cardiac insufficiency, cirrhosis of the liver, AIDS, age-related cachexia, acute and chronic infections, burns, COPD, chronic kidney insufficiency, malaria, hypophysial cachexia, cachexia suprarenalis, or Addison's disease.

In particular, the use of a PDE4 inhibitor or a PDE3/4 inhibitor or a pharmaceutically acceptable derivative thereof for the manufacture of a pharmaceutical composition for the treatment of cachexia as a result of cancer is preferred.

According to this invention, cancer refers to a cancer selected from the group consisting of breast cancer, ovarian cancer, stomach cancer, endometrial cancer, salivary gland cancer, lung cancer, kidney cancer, colon cancer, colorectal cancer, thyroid cancer, pancreatic cancer, prostate cancer and bladder cancer.

In a further embodiment of this invention, there is provided the use of a PDE4 inhibitor or a PDE3/4 inhibitor or a pharmaceutically acceptable derivative thereof for the manufacture of a pharmaceutical composition for the suppression of cytokines involved in the induction of a cachectic symptom.

According to this invention, suppression of cytokines refers to decreasing the elevated serum concentration of cytokines (i.e. TNF-α, IL-1, IL-6, IFN-γ, LIF or CNTF) in patients suffering from cachexia towards the concentration of said cytokines measurable in healthy humans.

In accordance with this invention, a cachectic symptom refers to a symptom selected from the group consisting of weight loss, anorexia, loss of protein mass, loss of fat mass, muscle atrophy and gain in the production of body water.

In accordance with this invention, PDE4 inhibitors or PDE3/4 inhibitors or pharmaceutically acceptable derivatives thereof are used for the preparation of a pharmaceutical composition. Therefore, PDE4 or PDE3/4 inhibitors may be part of a pharmaceutical composition, a pharmaceutical product or a preparation, and may be used in admixture with one or more pharmaceutically acceptable auxiliaries and/or excipients.

The person skilled in the art is familiar with pharmaceutical compositions, a pharmaceutical products or preparations and therefore, on the basis of his/her expert knowledge, the person skilled in the art knows which excipients or auxiliaries are suitable for the desired pharmaceutical composition, pharmaceutical product or preparation. In addition to solvents, gel-forming agents, tablet excipients and other active compound carriers, the person skilled in the art knows to use, for example, antioxidants, dispersants, emulsifiers, antifoams, flavour corrigents, preservatives, solubilizers, colorants or permeation promoters and complexing agents (e.g. cyclodextrins).

According to the present invention, a pharmaceutical composition comprising a PDE4 inhibitor or PDE3/4 inhibitor for the treatment of cachexia is administered orally, parenterally, intravenously, or percutaneously. In particular, oral administration and intravenous administration are preferred.

In case of a pharmaceutical composition (the term “pharmaceutical composition” is herein synonymous to pharmaceutical preparation), which is intended for oral administration, the therapeutic agent is formulated to give a medicament according to processes known per se and familiar to the person skilled in the art. The therapeutic agent is employed as medicament, preferably in combination with suitable pharmaceutical carriers, in the form of tablets, coated tablets, capsules, emulsions, suspensions or solutions, whereby the PDE4 inhibitor or PDE3/4 inhibitor content advantageously is between 0.1 and 95%, preferably between 1 and 80%, particularly preferred between 5 and 50%. By appropriate choice of the excipients and the auxiliaries it is possible to achieve a pharmaceutical administration form precisely tailored to the active ingredient(s) and/or to the desired onset of action (e.g. a sustained release form or an enteric form).

Injectable preparations, for example, sterile injectable aqueous or oleaginous solutions/suspensions can be formulated according to the known art using suitable dispersing or wetting agents and suspending agents. The sterile injectable preparation can also be a sterile injectable solution or suspension in a nontoxic parenteraly acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. Furthermore, fatty acids, such as oleic acid find use in the preparation of injectables. Dimethyl acetamide, surfactants including ionic and non-ionic detergents, polyethylene glycols can be used.

In general, satisfactory results will be obtained when the total daily dosage of the PDE4 or the PDE3/4 inhibitors, when taken oral or intravenous is in the range from 1-2000 μg/kg of body weight. In the case of the particularly preferred PDE4 inhibitor ROFLUMILAST, the daily dosage is in a range from 1-20 μg/kg of body weight. The daily dosage for the particularly preferred PDE3/4 inhibitor PUMAFENTRINE is in a range from 300-1500 μg/kg of body weight.

In case of oral administration of 3-cyclopropylmethoxy-4-difluoromethoxy-N-(3,5-dichloropyrid-4-yl)-benzamide (ROFLUMILAST), the adult daily dose is in the range from 50-1000 μg, preferably in the range from 250-500 μg, preferably by once daily administration.

In case of intravenous administration of 3-cyclopropylmethoxy-4-difluoromethoxy-N-(3,5-dichloropyrid-4-yl)benzamide (ROFLUMILAST), the adult daily dose is in the range from 50-600 μg, preferably in the range from 150-300 μg.

In a further embodiment of this invention, there is provided the use of a PDE4 inhibitor or a PDE3/4 inhibitor or a pharmaceutically acceptable derivative thereof for the manufacture of a pharmaceutical composition for enlarging survival period of a cancer patient afflicted to cachexia.

In an further embodiment of this invention, there is provided a method for treating a human afflicted to cachexia characterized by administration of a pharmaceutical composition comprising a PDE4 inhibitor or a PDE3/4 inhibitor or a pharmaceutically acceptable derivative thereof.

In a further embodiment of this invention, there is provided a method for treating a human afflicted to cachexia characterized by administration of a pharmaceutical composition comprising a PDE4 inhibitor or a PDE3/4 inhibitor or a pharmaceutically acceptable derivative thereof, whereby cachexia is a result of cancer, AIDS, age-related cachexia, acute and chronic infections, burns, chronic cardiac insufficiency, cirrhosis of the liver, COPD or chronic kidney insufficiency.

In a further embodiment of this invention, there is provided a method for treating a human afflicted to cachexia characterized by administration of a pharmaceutical composition comprising a PDE4 inhibitor or a PDE3/4 inhibitor or a pharmaceutically acceptable derivative thereof, whereby the PDE4 inhibitor or the PDE3/4 inhibitor or the pharmaceutically acceptable derivative thereof is effective to suppress cytokines involved in the induction of a cachectic symptom.

In a further embodiment of this invention, there is provided a method for the treatment of cachexia in a human afflicted to cancer comprising the step of administering an effective amount of a PDE4 inhibitor or a PDE3/4 inhibitor or a pharmaceutically acceptable derivative thereof.

In a further embodiment of this invention, there is provided a method for the treatment of cachexia in a human afflicted to cancer comprising the step of administering an effective amount of a PDE4 inhibitor or a PDE3/4 inhibitor or a pharmaceutically acceptable derivative thereof, whereby the survival period of a cancer patient afflicted to cachexia is enlarged.

In a further embodiment of this invention, there is provided a method for improving the response to chemo- and/or radiation-therapy in a human afflicted to cancer and cachexia comprising the steps of administering an effective amount of a chemotherapeutic agent and/or radiation and an effective amount of a PDE4 inhibitor or a PDE3/4 inhibitor or a pharmaceutically acceptable derivative thereof.

According to this invention, improving the response to chemo- and/or radiation therapy in a human afflicted to cancer and cachexia refers to prolonging the survival period of said human. In particular it refers to increasing the interval of time a human afflicted to cancer and cachexia survives after having chemo- and/or radiation therapy.

According to this invention, a PDE4 inhibitor or a PDE3/4 inhibitor or a pharmaceutically acceptable derivative thereof may be administered before, during and/or after radiation. It may be also administered before and during, during and after, before and after, or before, during and after radiation.

According to this invention, the source of radiation can be external or internal to the human treated. Radiation is administered in accordance with known techniques known to a person skilled in the art, such as external beam radiation therapy or brachytherapy, i.e. a therapy carried out by placing the source of radiation in the human. The dose of radiation depends on numerous factors as is well known in the art. Such factors include the organ being treated, the healthy organs in the path of the radiation that might be adversely affected, the tolerance of the patient for radiation therapy, and the area of the body in need of treatment. The dose will typically between 1 and 100 Gy, and more particular between 2 and 80 Gy.

According to this invention, chemotherapy refers to treatment with a chemotherapeutic agent. Therefore and in accordance with this invention, a PDE4 inhibitor or a PDE3/4 inhibitor or a pharmaceutically acceptable derivative thereof may be administered before, during, after, before and during, during and after, before and after, or before, during and after treatment with a chemotherapeutic agent.

According to this invention, chemotherapeutic agent is a chemotherapeutic drug selected from the group consisting 5 FU, actinomycin D, ABARELIX, ABCIXIMAB, ACLARUBICIN, ADAPALENE, ALEMTUZUMAB, ALTRETAMINE, AMINOGLUTETHIMIDE, AMIPRILOSE, AMRUBICIN, ANASTROZOLE, ANCITABINE, ARTEMISININ, AZATHIOPRINE, BASILIXIMAB, BENDAMUSTINE, BICALUTAMIDE, BLEOMYCIN, BROXURIDINE, BUSULFAN, CAPECITABINE, CARBOPLATIN, CARBOQUONE, CARMUSTINE, CETRORELIX, CHLORAMBUCIL, CHLORMETHINE, CISPLATIN, CLADRIBINE, CLOMIFENE, CYCLOPHOSPHAMIDE, DACARBAZINE, DACLIZUMAB, DACTINOMYCIN, DAUNORUBICIN, DESLORELIN, DEXRAZOXANE, DOCETAXEL, DOXIFLURIDINE, DOXORUBICIN, DROLOXIFENE, DROSTANOLONE, EDELFOSINE, EFLORNITHINE, EMITEFUR, EPIRUBICIN, EPITIOSTANOL, EPTAPLATIN, ERBITUX, ESTRAMUSTINE, ETOPOSIDE, EXEMESTANE, FADROZOLE, FINASTERIDE, FLOXURIDINE, FLUCYTOSINE, FLUDARABINE, FLUOROURACIL, FLUTAMIDE, FORMESTANE, FOSCARNET, FOSFESTROL, FOTEMUSTINE, FULVESTRANT, GEFITINIB, GEMCITABINE, GLIVEC, GOSERELIN, GUSPERIMUS, HERCEPTIN, IDARUBICIN, IDOXURIDINE, IFOSFAMIDE, IMATINIB, IMPROSULFAN, INFLIXIMAB, IRINOTECAN, LANREOTIDE, LETROZOLE, LEUPRORELIN, LOBAPLATIN, LOMUSTINE, MELPHALAN, MERCAPTOPURINE, METHOTREXATE, METUREDEPA, MIBOPLATIN, MIFEPRISTONE, MILTEFOSINE, MIRIMOSTIM, MITOGUAZONE, MITOLACTOL, MITOMYCIN, MITOXANTRONE, MIZORIBINE, MOTEXAFIN, NARTOGRASTIM, NEBAZUMAB, NEDAPLATIN, NILUTAMIDE, NIMUSTINE, OCTREOTIDE, ORMELOXIFENE, OXALIPLATIN, PACLITAXEL, PALIVIZUMAB, PEGASPARGASE, PEGFILGRASTIM, PENTETREOTIDE, PENTOSTATIN, PERFOSFAMIDE, PIPOSULFAN, PIRARUBICIN, PLICAMYCIN, PREDNIMUSTINE, PROCARBAZINE, PROPAGERMANIUM, PROSPIDIUM CHLORIDE, RALTITREXED, RANIMUSTINE, RANPIRNASE, RASBURICASE, RAZOXANE, RITUXIMAB, RIFAMPICIN, RITROSULFAN, ROMURTIDE, RUBOXISTAURIN, SARGRAMOSTIM, SATRAPLATIN, SIROLIMUS, SOBUZOXANE, SPIROMUSTINE, STREPTOZOCIN, TAMOXIFEN, TASONERMIN, TEGAFUR, TEMOPORFIN, TEMOZOLOMIDE, TENIPOSIDE, TESTOLACTONE, THIOTEPA, THYMALFASIN, TIAMIPRINE, TOPOTECAN, TOREMIFENE, TRASTUZUMAB, TREOSULFAN, TRIAZIQUONE, TRIMETREXATE, TRIPTORELIN, TROFOSFAMIDE, UREDEPA, VALRUBICIN, VERTEPORFIN, VINBLASTINE, VINCRISTINE, VINDESINE, VINORELBINE and VOROZOLE.

### FIGURES

FIG. 1: Effects of Zardaverine (left graph) and Piclamilast (right graph) on the secretion of IL-1 from LXFA 526 lung adenocarcinoma Xenograft explants. Tumor fragments from mice carrying LXFA 526 xenografts were excised and cell suspensions were seeded into 24 Well plates. After seeding of the cells, Zardaverine at concentrations of 100, 3, and 0.1 μM, respectively, and Piclamilast at concentrations of 1, 0.3 and 0.001 μM, respectively were added and supernatants were harvested 24 hours later. IL-1 content of the supernatants was quantitated using R&D Quantikine ELISA kits according to the manufacturer's recommendations. Zardaverine (left graph) was able to suppress the secretion of IL-1 from 85.3 pg/ml in control supernatants (treated with the respective amount of DMSO) to 64.5; 67.9, and 77.1 pg/ml at concentrations of 100, 3, and 0.1 μM, respectively. Piclamilast (right graph) was able to suppress the secretion of IL-1 from 85.3 pg/ml in control supernatants (treated with the respective amount of DMSO) to 68.9; 64.4, and 81.9 pg/ml at concentrations of 1; 0.3, and 0.001 μM, respectively.

FIG. 2: Effects of Zardaverine (left graph) and Piclamilast (right graph) on the secretion of IL-1 from RXF 393 hypernephroma Xenograft explants. Tumor fragments from mice carrying RXF 393 xenografts were excised and cell suspensions were seeded into 24 Well plates. After seeding of the cells, Zardaverine at concentrations of 100 and 0.1 μM, respectively, and Piclamilast at concentrations of 1, 0.01 and 0.001 μM, respectively were added and supernatants were harvested 24 hours later. IL-1 content of the supernatants was quantitated using R&D Quantikine ELISA kits according to the manufacturer's recommendations. Zardaverine (left graph) was able to suppress the secretion of IL-1 from 46.9 pg/ml in control supernatants (treated with the respective amount of DMSO) to 32.1 and 48.1 pg/ml at concentrations of 100 and 0.1 μM, respectively. Piclamilast (right graph) was able to suppress the secretion of IL-1 from 46.9 pg/ml in control supernatants (treated with the respective amount of DMSO) to 37.8; 43.9, and 43.2 pg/ml at concentrations of 1; 0.01, and 0.001 μM, respectively.

FIG. 3: Effects of Zardaverine (left graph) and Piclamilast (right graph) on the secretion of TNFα from LXFA 526 lung adenocarcinoma Xenograft explants. Tumor fragments from mice carrying LXFA 526 xenografts were excised and cell suspensions were seeded into 24 Well plates. After seeding of the cells, Zardaverine at concentrations of 100, 3, and 0.1 μM, respectively, and Piclamilast at concentrations of 1, 0.3 and 0.001 μM, respectively were added and supernatants were harvested 24 hours later. TNFα content of the supernatants was quantitated using R&D Quantikine ELISA kits according to the manufacturer's recommendations. Zardaverine (left graph) was able to suppress the secretion of TNFα from 16.8 pg/ml in control supernatants (treated with the respective amount of DMSO) to 12.6; 12.9, and 13.8 pg/ml at concentrations of 100, 3, and 0.1 μM, respectively. Piclamilast (right graph) was able to suppress the secretion of IL-1 from 16.8 pg/ml in control supernatants (treated with the respective amount of DMSO) to 13.9; 15.1, and 17.2 pg/ml at concentrations of 1; 0.3, and 0.001 μM, respectively.

FIG. 4: Effects of Zardaverine (left graph) and Piclamilast (right graph) on the secretion of TNFα from LXFE 397 epidermoid adenocarcinoma Xenograft explants. Tumor fragments from mice carrying LXFE 397 xenografts were excised and cell suspensions were seeded into 24 Well plates. After seeding of the cells, Zardaverine at concentrations of 30, 10, and 3 μM, respectively, and Piclamilast at concentrations of 0.3; 0.1; and 0.03 μM, respectively were added and supernatants were harvested 24 hours later. TNFα content of the supernatants was quantitated using R&D Quantikine ELISA kits according to the manufacturers recommendations. Zardaverine (left graph) was able to suppress the secretion of TNFα from 30.3 pg/ml in control supernatants (treated with the respective amount of DMSO) to 20.1; 28.2, and 27.7 pg/ml at concentrations of 30, 10, and 3 μM, respectively. Piclamilast (right graph) was able to suppress the secretion of TNFα from 21.5 pg/ml in control supernatants (treated with the respective amount of DMSO) to 10; 12.1, and 14.1 pg/ml at concentrations of 0.3, 0.1; and 0.03 μM, respectively.

### EXAMPLE

Effectiveness of PDE3/4 and PDE4 inhibitors in the suppression of cytokines associated with the onset of cachexia.

The PDE3/4 inhibitors Zardaverine and PDE4 inhibitor Piclamilast were utilized in order to generally exemplify the suitability of PDE4 and/or PDE3/4 inhibitors in the suppression of cachexia-inducing cytokines.

To this end primary cultures of tumor cells were derived from cachexia inducing human xenografts grown subcutaneously in nude mice. The xenograft cell derivatives and their measurable cytokine secretions are summarized in Table 1.

TABLE 1

| | | | TNFα |
|---|---|---|---|
| Tumor | Histology | IL-1 | comment |
| RXF393 | hypernephroma | + | + |
| LXFA 526 PXC | adenocarcinoma (lung) | + | + |
| LXFE397 | epidermoid | not detectable | + |

5 to 10 NMRI nude mice were implanted with tumor fragments derived from the above mentioned Xenografts and grown until the tumors reached approximately 0.5 g, which correlated well with the onset of cachexia. Animals were then sacrificed and tumors were excised. Cells were subsequently isolated under sterile conditions by the use of mechanic disintegrators, proteases, hyaluronidase, and DNAse I. The crude suspension was passed twice through sterile sieves with diameters of 200 and 50 μM, respectively.

Washed cell pellets were resuspended in Iscove's modified Dulbecco's Medium +20% FCS (fetal calf serum) and 0.24 to 1×10⁶ tumor cells were seeded in 24 well plates. The cell isolates not only contained tumor cells, but also blood cells and stromal elements of murine origin. The cell lines RXF 486L was directly utilized and 1×10⁶ cells were seeded into each well.

Piclamilast and Zardaverine were dissolved in 100% DMSO (dimethyl sulfoxide) and applied at a final concentration of 1 μM to 0.001 μM (Piclamilast) or 100 μM to 0.1 μM (Zardaverine). Drugs and cells were plated at the same time. 24 hours after seeding supernatants were collected, centrifuged, and stored at −80° C.

For the quantitative measurement of IL-1, IL-6, and TNFα, respectively, the supernatants were analyzed using Quantikine ELISA Kits from R&D Systems according to the manufacturer's recommendations.

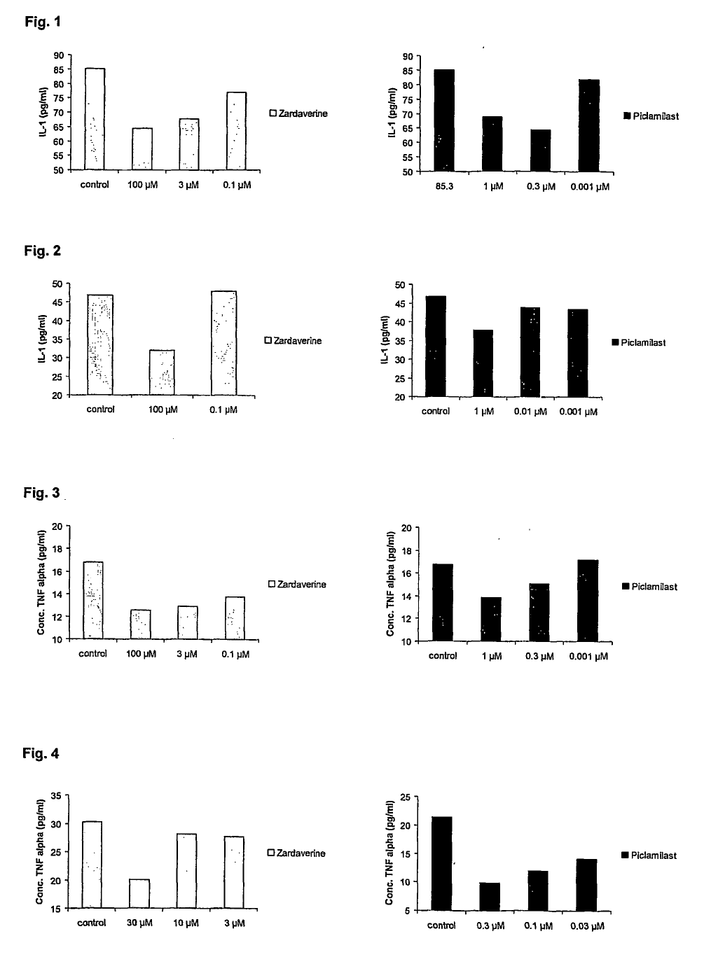

Modulation of IL-1 expression was investigated in two cell systems: LXFA 526, and RXF 393. Both, Zardaverine and Piclamilast were in both cell lines able to suppress IL-1 levels with Zardaverine being slightly more active than Piclamilast (results are shown in FIGS. 1 and 2).

The modulation of TNFα levels by Zardaverine and Piclamilast was investigated in LXFA 526 cells and in the LXFE 397 model, as shown in FIGS. 3 and 4. In both cell systems, Zardaverine and Piclamilast were able to inhibit the secretion of TNFα by the respective cell isolates into the medium.

These data show that PDE4 as well as PDE 3/4 inhibitors have the potential to suppress the secretion of cytokines linked to cachexia from tumor cell isolates that originate from cachexia-inducing xenografts. It has to be noted that COX-2 inhibitors that to some extent exert anti-cachectic activity depending on the model system investigated, were unable in this system to suppress the secretion of either IL-1 or TNFα to any significant extent in the above described system.

1. A method for treating cachexia, comprising administering to a patient in need thereof a thereapeutically effective amount of a PDE4 inhibitor or a pharmaceutically acceptable derivative thereof, or a PDE3/4 inhibitor or a pharmaceutically acceptable derivative thereof.

2. The method according to claim 1, whereby the cachexia is a result of cancer, AIDS, age, acute and chronic infections, burns, chronic cardiac insufficiency, cirrhosis of the liver, COPD or chronic kidney insufficiency.

3. The method according to claim 2, wherein the cachexia is a result of cancer.

4. The method according to claim 3, wherein the cancer is selected from the group consisting of breast cancer, ovarian cancer, stomach cancer, endometrial cancer, salivary gland cancer, lung cancer, kidney cancer, colon cancer, colorectal cancer, thyroid cancer, pancreatic cancer, prostate cancer and bladder cancer.

5. The method as claimed in claim 1, wherein the PDE4 inhibitor or PDE3/4 inhibitor is selected from the group consisting of CDC-998, SH-636, D-4396, SCH-351591, IC-485, CC-1088, KW-4490 and 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamideoxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, (−)-cis-9-ethoxy-8-methoxy-2-methyl-1,2,3,4,4a, 10b-hexahydro-6-(4-diisopropylamino-carbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [Research-Code: BAY-19-8004], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST], 3-Cyclopropylmethoxy-4-difluoromethoxy-N-(3,5-dichloropyrid-4-yl)-benzamide [INN: ROFLUMILAST], and pharmaceutically acceptable derivatives thereof.

6. The method as claimed in claim 1, wherein the PDE4 inhibitor or PDE3/4 inhibitor is selected from the group consisting of (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE] 3-Cyclo-propylmethoxy-4-difluoromethoxy-N-(3,5-dichloropyrid-4-yl)-benzamide [INN: ROFLUMILAST], and pharmaceutically acceptable derivatives thereof.

7. The method as claimed in claim 1, wherein the PDE3/4 inhibitor is (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE] or a pharmaceutically acceptable derivative thereof.

8. The method as claimed in claim 1, wherein the PDE4 inhibitor is 3-Cyclopropylmethoxy-4-difluoromethoxy-N-(3,5-dichloropyrid-4-yl)-benzamide [INN: ROFLUMILAST] or a pharmaceutically acceptable derivative thereof.

9. The method as claimed in claim 8, wherein the PDE4 inhibitor is 3-Cyclopropyl-methoxy-4-difluoromethoxy-N-(3,5-dichloropyrid-4-yl)-benzamide [INN: ROFLUMILAST] or a pharmaceutically acceptable salt or solvate thereof, or a pharmaceutically acceptable N-oxide or a pharmaceutically acceptable salt or solvate of the latter.

10.-12. (canceled)

13. The method according to claim 1, wherein the PDE4 inhibitor or the pharmaceutically acceptable derivative thereof, or the PDE3/4 inhibitor or the pharmaceutically acceptable derivative thereof is effective to suppress cytokines involved in the induction of a cachectic symptom.

14. A method for treating cachexia in a human afflicted with cancer comprising the step of administering to a patient in need thereof a therapeutically effective amount of a PDE4 inhibitor or a pharmaceutically acceptable derivative thereof, or a PDE3/4 inhibitor or a pharmaceutically acceptable derivative thereof.

15. The method according to claim 14, whereby the survival period of a cancer patient afflicted to cachexia is enlarged.

16. A method for improving the response to chemo- and/or radiation-therapy in a human afflicted to cancer and cachexia comprising the steps of administering an effective amount of a chemotherapeutic agent and/or radiation and an effective amount of a PDE4 inhibitor or a pharmaceutically acceptable derivative thereof, or a PDE3/4 inhibitor or a pharmaceutically acceptable derivative thereof.
